SEQUENCE LISTING (1 sequence)

SEQ ID NO: 1 - DNA, 11155 bp, synthetic construct
  source 1..11155: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgcccaagctctacaacggagtctacagcggtcagtgcggcgccctatcgccacctgacctcatggaggcccagccgaagctacttcccaagccaaggagcaacagcagcggcagcaccggccggaacagcaagtattggatattttcaatgataatcgagcgcagtgcgggtcccaagcggattgaaatcgatggcgatgatgcggacacgccgctggaggccatcctgccagccgaaccgccggcggaggtctgcctcttgcgtgacagccccttcaggatattgcgggcggctgagtccggaaaccttgacgacttcaagcgactcttcatggcggacaactcgcgcattgctttaaaggatgcgaaaggacgaacggctgcccatcaggcggcggcccgtaatagggttaacattttgcggtacattcgcgaccagaatggcgactttaatgcgaaggataatgccggcaataccccgctccacatcgccgtggagagcgatgcctacgacgctctggactatctattgtccatcccagtggatacgggagtgctgaacgagaagaagcaggcaccagtgcacttggccaccgagctgaacaaagtgaagtcccttcgggtgatgggtcagtaccgcaatgtcatcgatattcagcagggcggcgaacatggacgtaccgctctgcacttggccgccatctatgatcacgaggagtgcgctcgcatcctgataactgagttcgatgcatgcccacgtaagccctgtaacaatggttattatcccatacacgaagcggccaagaatgccagctccaagacaatggaggtcttcttccagtggggcgagcagcgcggctgcacccgcgaggagatgatatccttctacgactcggagggcaatgtgccgctccattcggctgtccatggtggcgacatcaaggctgtggagctgtgcctcaagtccggggccaagatatctacgcagcaacacgatctctcgacgccagtgcacctggcttgtgcccagggagccatagacattgtaaagctcatgttcgagatgcagccaatggagaagcgactatgtctgagttgcacggatgtgcagaagatgacgccgctgcactgcgcctccatgttcgatcatccggacattgtgtcctatctggtggccgagggagcggacatcaatgccctggacaaggagcatcgctctccgttactgctggcggcatctcgtagcggttggaaaacagtccacctcctgattcgcctgggggcgtgcattagtgtcaaggacgccgccgcccgcaatgtgctgcacttcgtcatcatgaacggcggccggctgacggacttcgcggagcaggtggccaattgccagacgcaggcgcagctgaagctgctgctcaacgagaaggacagcatgggctgctcgccgctgcactacgccagtcgggatggacacatccgttcgttggagaacctcattcgactgggagcctgcatcaacctgaagaacaacaacaacgagagtccgctgcactttgccgcacgctacggaagatacaatacggtgcggcagctcttggattccgagaagggatccttcatcatcaacgaaagtgacggtgcagggatgacaccactgcacatatcctcgcagcaaggacacacgcgagtggtgcagctgctactcaatcgaggagccctgctccatcgggaccacaccggacgcaatcctctccagctagcggccatgtccggatacaccgagaccatcgagctgctgcactcggtgcactcgcatctgctcgatcaggtggacaaggatgggaacaccgctcttcacctggccaccatggagaataagccccatgcgatctccgtgctgatgtctatgggctgtaagctggtctataacgttctggacatgagtgccattgactatgccatctactacaaatatccggaggctgccctggccatggtcacccacgaggagcgggccaacgaggtgatggctctgcgttccgacaagcatccgtgcgtgaccctcgccttaattgcctccatgcccaaggtattcgaggcggtgcaggacaagtgcattaccaaggccaattgcaagaaggactcgaagagtttctacataaaatattcgttttggccctaccaaaagacacccgaacagattgaggccaagcgcaaagagttcaatgaccccaagtggcgacccgcgcctttggccgtggtgaacaccatggtaacacatggcagggtggagctgctggcccatccgctcagtcagaagtatctgcagatgaagtggaactcctacggcaagtactttcacctggccaacctgctaatctactcgatattcctggtctttgtaaccatctactcttcgctgatgatgaacaacatcgaactgaaggctggggacaacaagacgatgagtcaatactgcaatatgggatgggagcagctgaccatgaatctctcgcagaacccgtcggtggcatcacagattcgtttggattcctgcgaggagcgtataaatagaaccactgcaatacttttctgtgcggtggtcatcgtggtctatatactgctcaactcgatgcgggaactaatacagatataccagcagaaattgcactatatcctggagacagttaatttgatatcctgggtgctgtacatctcggctttggtgatggtaacaccggcatttcagccggatggaggaatcaataccattcattactcggccgcttcaatagcagtctttctgtcgtggttccgattgctactgttcctgcaaagattcgaccaggtcggcatctatgtggtcatgttcttggagattctgcagacgctcattaaagtgctgatggtattctccatacttataatcgcctttggtctggctttctatatactactttcaaagattattgacccccaaccgaaccacttgtccttctccaacatacccatgtccttgctgcgaactttctcaatgatgctgggcgagctggactttgtgggtacctatgtgaacacctactatcgggatcagttgaaagtgcccatgacatcctttttgattttgagtgtctttatgatccttatgcccattcttctgatgaacttgctcatcggtttggccgtcggcgatattgagtcagtgcgtcgcaatgcccagctcaagagactggccatgcaggtggtgctccacacggagctggagaggaagttgccccatgtctggctgcagcgagttgacaagatggagctgattgagtatcccaatgaaaccaagtgcaagctgggcttctgcgatttcatcctgcgcaagtggttctcgaatccattcaccgaggattcctccatggacgtcatctccttcgacaacaatgatgactacatcaacgcagaattggaacggcagaggcgaaagttgcgcgacataagtcgcatgctggagcaacagcaccatctggttcggctcattgtccaaaagatggagatcaagacggaggcggatgacgtggacgagggtatatccccaaacgagttgcgatccgtcgtcggtttgagatcggcaggcggaaatcgatggaactcgccgcgagtccggaataaactccgagccgccctgagcttcaataagagcatggaacaaaaactcatctcagaagaggatctgggaagcggagccacgaacttctctctgttaaagcaagcaggagatgttgaagaaaaccccgggcctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

(19) United States
(12) Patent Application Publication
(10) Pub. No.: US 2025/0367323 A1
(43) Pub. Date: Dec. 4, 2025

(54) PLATFORM FOR RADIOLYTIC NEUROMODULATION

(71) Applicant: THE UAB RESEARCH FOUNDATION, BIRMINGHAM, AL (US)

(72) Inventors: Aurelio Galli, Brentwood, TN (US); Mark Bolding, Birmingham, AL (US); Steven Rothenberg, Vestavia Hills, AL (US); Jennifer DeBerry, Birmingham, AL (US); Angela Carter, Birmingham, AL (US)

(21) Appl. No.: 19/202,036

(22) Filed: May 8, 2025

Related U.S. Application Data
(60) Provisional application No. 63/654,217, filed on May 31, 2024.

(51) Int. Cl. A61K 48/00; A61N 5/10; C12N 15/86

(52) U.S. Cl. CPC A61K 48/0058; A61N 5/1077; C12N 15/86; A61N 2005/1098; C12N 2740/15043

(57) ABSTRACT

In one aspect, the disclosure relates to a method for neuromodulation in a target, wherein the target can be an isolated plurality of cells, an isolated organ, or an animal subject In an aspect, the method includes at least the step of delivering X-ray radiation to one or more neurons in the target, wherein the one or more neurons express dTRPA1(A)10b, and wherein the X-ray radiation generates H₂O₂ via radiolysis of water. In a further aspect, the H₂O₂ can increase intracellular calcium ions which can, in turn, stimulate the release of neurotransmitters. Also disclosed are treatments of diseases, disorders, and symptoms thereof using the disclosed method.

### CROSS REFERENCE TO RELATED APPLICATIONS

This application claims the benefit of U.S. Provisional Application Ser. No. 63/654,217, filed May 31, 2024, which is incorporated herein by reference in its entirety.

### CROSS-REFERENCE TO SEQUENCE LISTING

This application contains a sequence listing filed in ST.26 format entitled “222120-2070_Sequence_Listing.xml” created on Apr. 10, 2025, and having a file size of 13,148 bytes. The content of the sequence listing is incorporated herein in its entirety.

### BACKGROUND

Neuromodulation encompasses interventions and technologies that deliver stimuli to peripheral or central nervous system targets to achieve a desired effect. Neuromodulation technologies currently on the market include: neurofeedback, entrainment, focused ultrasound, transcranial direct current stimulation, transcranial alternate current stimulation, cranial nerve stimulation (e.g. vagus nerve stimulation), transcranial magnetic stimulation, peripheral nerve stimulation, implantable devices (e.g. spinal cord stimulation or deep brain stimulation), and chemo/optogenetics that regulate the activity of genetically defined neurons and/or brain regions. However, the effectiveness of each of these technologies is dampened by one or more factors including a lack of regional/neuronal specificity, the requirement of permanent implants, being restricted to an illuminated region of the nervous system, and/or limited control of stimulus intensity.

Biological techniques that enable the regulation of the activity of genetically defined neurons provide opportunities for examining how circuitries control specific behaviors. In particular, optogenetics allows the control of activities of individual neurons and release of neurotransmitters in living tissue, even within freely moving animals. However, it has several limitations, including requiring permanent intracranial implants, being restricted to the illuminated region of the brain, and limited regulation of the stimulation intensity.

It would be desirable to develop a non-invasive approach, with controlled stimulus intensity, capable of regulating cell activity and fate with regional/neuronal specificity. This approach would enable normalization of distinct neurocircuits involved in the pathophysiology of neuropsychiatric disorders, correct behaviors associated with aberrant activity of specific neuronal populations in neurological disorders, and control dysregulated neuronal cell growth (e.g. cancer). These needs and other needs are satisfied by the present disclosure.

### SUMMARY

In accordance with the purpose(s) of the present disclosure, as embodied and broadly described herein, the disclosure, in one aspect, relates to a method for neuromodulation in a target, wherein the target can be an isolated plurality of cells, an isolated organ, or an animal subject. In an aspect, the method includes at least the step of delivering X-ray radiation to one or more neurons in the target, wherein the one or more neurons express dTRPA1(A)10b, and wherein the X-ray radiation generates H₂O₂ via radiolysis of water. In a further aspect, the H₂O₂ by activating dTRPA1(A)10b can allow ion fluxes through the plasma membrane causing membrane depolarization and increase intracellular calcium ions which can, in turn, stimulate the release of neurotransmitters. Also disclosed are treatments of diseases, disorders, and symptoms thereof using the disclosed method.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show X-ray irradiation induces increases in intracellular Ca²⁺ in a dTRPA1(A)10b-dependent manner. (FIG. 1A) Cells expressing either dTRPA1, mTRPA1, or mCherry were Xray irradiated (0.5 nGy/cm²/sec) for 60 sec while monitoring changes in intracellular Ca²⁺. BITC (100 μM) was added at the end of the experiments to confirm TRPA1 expression. (F_{(2,87)}=66.35; p≤0.0001); dTRPA1(A)10b vs. mTRP1; n=25-57. (FIG. 1B) As in (FIG. 1A), except cells were stimulated with H₂O₂ (10 μM) administration. (F_{(2,87)}=62.76; p≤0.0001); dTRPA1(A)10b vs. mTRPA1; n=42-51. One-way ANOVA with Tukey's multiple comparisons test.

FIGS. 2A-2C show dTRPA1(A)10b expressed in cortical neurons enables Xrs-induced depolarization and action potential firing. (FIG. 2A) Whole-cell current clamp recording from cortical neurons (picture, scale bar 15 μm) expressing dTRPA1(A)10b/mCherry. X-ray stimulation (Xrs on) causes robust depolarization and action potential firing. (FIG. 2B) Current clamp recording from cortical neurons expressing mCherry only (picture). Xray stimulation failed to elicit substantial membrane depolarization. However, depolarizing current injections caused action potential firing (inset). (FIG. 2C) Membrane depolarization (ΔV) caused by Xray in mCherry and dTRPA1(A)10b positive (+) neurons. (Student t-test; ***=p<0.0004; n=5-8).

FIGS. 3A-3D show development of X-rays as a technology to stimulate neurotransmitter release and associated behaviors. (FIG. 3A) Image of the Xrs source including servo-controlled lead shuffler (Moxtek MAGPRO 60 kV; 12 W). (FIG. 3B) (top) A Drosophila brain stained for tyrosine hydroxylase (α-TH) to mark DA neurons, and Bruchpilot (α-nc82), an active zone marker. The amperometric electrode (5 μm) is placed in proximity to the PML of the MB (white circle) to record DA release. Below is shown arborization of a single DA PPL1 neuron (PPL101, yellow) projecting to the PML of the MB. Xrs irradiation causes H₂O₂ synthesis, dTRPA1 channel stimulation (middle) and, as a consequence, DA release (bottom). This data also points to the Drosophila TR PA1 channel (dTRPA1) expressed in DA neurons as the mediator of Xrs stimulated DA release. (FIG. 3C) In Drosophila, Xrs irradiation promotes locomotion, and grooming, DA-associated behaviors. (FIG. 3D) Future Directions: This data demonstrates that Xrs stimulate DA release in Drosophila brain, but not in murine striatal slices, a failure that is attributed to the absence of dTRPA1 channels. Therefore, once the role of dTRPA1 channels for Xrs stimulated DA release and behaviors is further delineated, the dTRPA1(A)10b (a thermo-insensitive isoform) will be virally expressed in specific nodes/neurons in mice to cause selective Xrs mediated neurotransmitter release and associated behaviors.

FIGS. 4A-4G show Xrs-stimulated vesicular DA release is TTX-sensitive and TRPA1-dependent. (FIG. 4A) Xrs stimulated (0.1 Gy/sec for 250 msec) DA release recorded from isolated Drosophila brain. (FIG. 4B) Incubation of fly brains with 1 μM TTX for 5 min blocked the ability of Xrs to stimulate DA release. (FIG. 4C) Quantitation of the area under the curve (AUC) of the amperometric current in the presence or absence of TTX (Student t-test; **=p<0.003; n=4-7). (FIG. 4D) Representative amperometric trace recorded as in (FIG. 4A) in the absence of Drosophila brain (representative of n=10). DA release recorded as in (FIG. 4A) from Drosophila brains incubated with either vehicle (FIG. 4E) or 100 μM HC-030031 (FIG. 4F) for 5 min. Quantitation of the AUC (FIG. 4G) of the amperometric currents in the presence or absence of HC-030031 (Student t-test; *=p<0.015; n=5-7).

FIGS. 5A-5C show TRPA1(A) RNAi in DA neurons inhibits Xrs-stimulated DA release. Xrs (0.1 Gy/sec; 250 msec) stimulated DA release from flies expressing either (FIG. 5A) TH-mCherry or (FIG. 5B) TH-TRPA1 RNAi. (FIG. 5C) Quantitation of AUC of amperometric signals (Student's t-test; *=p<0.02; n=8-10).

FIGS. 6A-6C show Xrs stimulation fails to cause DA release in mouse striatal slices. (FIG. 6A) DA release was electrically stimulated with a bipolar electrode (200 μA, 100 msec). (FIG. 6B) Xrs stimulation (0.1 Gy/sec; 250 msec) failed to elicit DA release. (inset). Zoom in on the artifact promoted by Xrs stimulation. (FIG. 6C) A second electrical stimulation, following the Xrs stimulation, caused DA release (representative of n=3).

FIGS. 7A-7B show H₂O₂ stimulates DA release. (FIG. 7A) Amperometric current recorded in absence (CTR) or presence of H₂O₂. (FIG. 7B) Current values recorded with electrode in or out of the brain (Student's t-test; in vs. out; **=p<0.0042; n=3).

FIGS. 8A-8B show Xrs stimulate DA-associated behaviors: (FIG. 8A) Total grooming time (20 sec period) before (Pre-Xrs) and during (During-Xrs) Xrs stimulation (Student's t-test; **=p<0.0025; n =11). (inset) Avg locomotor velocity of a representative fly pre-Xrs stimulation and during (arrow) Xrs stimulation. (FIG. 8B) Distance travelled Pre-Xrs (30 sec) and During-Xrs stimulation (0.2 Gy/sec; 30 sec); (Student's t-test; *=p<0.035; n =9).

FIG. 9 shows administration of H₂O₂ increases locomotion: H₂O₂ significantly increases total beam crossings over a 4 hr period with respect to control (Student's t-test; *=p<0.03; n=29-30).

FIGS. 10A-10C show differential ROS levels exist between glioblastoma cell lines. FIG. 10A: Relative basal ROS levels were measured, using the DCFDA-ROS assay, across six glioblastoma cells lines. The U251 line exhibited lower ROS levels than all other lines. The D54 line exhibited significantly higher ROS levels than LN229, U251, U87, and LN18 and trended slightly higher than U373. FIG. 10B: Representative fluorescence signal ratio over time in D54 (putative ROS-high) and U251 (putative ROS-low) cells transfected with the Hyper3 probe. FIG. 10C: Quantification of approximate basal ROS levels from traces collected as in b, using the formula [H₂O₂]=K_{d}×(F−Fₘᵢₙ)/(Fₘₐₓ−F), assuming K_{d}˜Kₒₓ. Kₒₓ for Hyper3=260 nM H₂O₂.

FIG. 11 shows a map of VSV-G pseudotyped third-generation lentivirus carrying dTRPA1(A)10b DNA and associated control. The vector was synthesized and packaged by Vectorbuilder. An exemplary vector has SEQ ID NO. 1.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In one aspect, disclosed herein is a non-invasive platform for radiolytic neuromodulation. In a further aspect, the platform and associated methods are customizable to have regional and/or neuronal specificity. In a still further aspect, the platform and associated methods are also customizable with respect to stimulus intensity. In an aspect, customization of the present platform and method allows for precise tailoring of the system and method to treat neuropsychiatric disorders, to treat symptoms associated with neuronal dysfunction, and to control dysregulated neuronal cell growth.

### Method for Neuromodulation in a Subject

Disclosed herein is a method for neuromodulation in a subject, the method including at least the steps of virally transfecting one or more neurons in the subject with a gene encoding dTRPA1(A)10b, thereby causing dTRPA1(A)10b channels to be expressed in the one or more neurons, and delivering X-ray radiation to the one or more neurons, wherein the X-ray radiation generates H₂O₂ via radiolysis of water and wherein the H₂O₂ activates the dTRPA1(A)10b channels; thereby increasing intercellular calcium and releasing one or more neurotransmitters. In some aspects, the disclosed method can be adapted to other receptors to achieve similar effects. In a further aspect, the receptors can be dTRPA1(A)10b analogues, such as, for example, mosquito-derived dTRPA1, other heat independent receptors, and/or receptors present on non-neuronal cell types. In still another aspect, the receptors can be natural receptors or can be sequence-modified receptors introduced to cells through viral transfection or other methods. In one aspect, in vivo transfection can be accomplished virally, while in vitro transfection can be accomplished virally, by a physical means to transfect neuronal cultures such as, for example, electroporation, microinjection, or biolistics, or by a chemical method such as, for example, Ca²⁺ phosphate co-precipitation or lipofection. In one aspect, the sequences of the receptors can be modified to incorporate temperature sensitive properties. Further in this aspect, the main thermal responsive elements for Drosophila TRPA1 can be found in the C-terminal cytosolic domain of the protein, specifically at exon 12. However, in a further aspect, no single domain of the dTRPA1 channel can completely explain the thermal-response properties of this protein. In one aspect, complex allosteric interactions likely depend on the context of intervening ankyrin repeats between the N- and C-termini. In a still further aspect, temperature sensitivity has been determined for all isoforms from about 15° C. to about 42° C. In one aspect, the viral transfection can be lentiviral transfection. Further in this aspect, lentiviral transfection is especially suitable due to constraints generated by the size of the TRPA1 DNA. However, other transfection methods are also contemplated and should be considered disclosed.

In an aspect, amounts of radiation useful in the disclosed method can vary but, in some cases, can be from about 1 mGy to about 500 mGy, or from about 10 mGy to about 250 mGy, or from about 100 mGy to about 150 mGy, or can be about 1, 5, 10, 25, 50, 100, 150, 200, 250, 300, 350, 400, 450, or about 500 mGy, or a combination of any of the foregoing values, or a range encompassing any of the foregoing values. In some aspects, the method can produce about 70 nM H₂O₂ per Gy of X-ray radiation delivered.

In another aspect, X-ray radiation exposure time can be from about 1 ms to about 25 s, from about 1 ms to about 5 s, or from about 5 ms to about 1 s, or from about 100 ms to about 500 ms, or can be about 1 ms, 50 ms, 100 ms, 500 ms, 1 s, 1.5 s, 2 s, 2.5 s, 3 s, 3.5 s, 4 s, 4.5 s, or about 5 s, or a combination of any of the foregoing values, or a range encompassing any of the foregoing values. In still another aspect, the X-ray radiation can be delivered at a frequency or dose rate of from about 0.1 Hz to about 100 Hz, or from about 0.5 Hz to about 50 Hz, or from about 1 Hz to about 10 Hz, or at about 0.1, 0.5, 1, 5, 10, 20, 30, 40, 45, 50, 55, 60, 65, 70, 75, 80, 85, 90, 95, or about 100 Hz, or a combination of any of the foregoing values, or a range encompassing any of the foregoing values. In some aspects, the radiation exposure time and dose can be higher or lower depending on the severity of the disease or symptom being treated.

In any of these aspects, the hydrogen peroxide induces an increased level of intracellular calcium ions compared to the level of intracellular calcium ions prior to performing the method. In a further aspect, the hydrogen peroxide can stimulate the release of at least one neurotransmitter. In one aspect, the neurotransmitter can be dopamine, norepinephrine, serotonin, or any combination thereof.

The disclosed method can be used in medical treatment or for research purposes. In one aspect, the target can be an isolated plurality of cells such as, for example, neurons. In one aspect, the neurons can be mammalian neurons that have been virally transfected with a gene that encodes dTRPA1(A)10b. In another aspect, the target can be an isolated organ such as, for example, a brain. In still another aspect, the target can be an animal subject including an invertebrate or a mammal. Further in this aspect, the radiation can be applied to at least one neuron in the animal subject. In one aspect, the invertebrate can be Drosophila. Further in this aspect, the invertebrate can express dTRPA1(A)10b naturally (endogenously). In an alternative aspect, the mammal can be a human, mouse, rat, guinea pig, hamster, rabbit, cat, dog, cattle, horse, swine, goat, sheep, or non-human primate that has been virally transduced with dTRPA1(A)10b or expressing dTRPA1(A)10b by genetic approaches. In one aspect, when the animal subject is a mammal, one or more neurons in the mammal can be virally transfected with dTRPA1(A)10b or expressed by genetic approaches prior to performing the method. In any of these aspects, the at least one neuron can be a neuron in the central nervous system, a neuron in the peripheral nervous system, or any combination thereof. In another aspect, this approach can be used to cause neuromodulation of specific neurocircuits and associate this activation to specific behaviors.

### Use of Nanoparticles

In some aspects, the disclosed method can be performed in the presence of metallic nanoparticles. Without wishing to be bound by theory, under ionizing radiation such as X-rays, metallic nanoparticles can be used to enhance the generation of ROS. In one aspect, the nanoparticles can be gold, platinum, bismuth, tungsten, silver, gadolinium, tantalum, or any combination thereof.

In another aspect, the nanoparticles can be metal oxide nanoparticles. Without wishing to be bound by theory, metal oxides are known to catalyze redox reactions and may have especially high activity under ionizing radiation. In one aspect, the metal oxide nanoparticles can be titanium dioxide (TiO₂) (having photo- and radiocatalytic properties), zinc oxide (ZnO), cerium oxide (CeO₂) (serving as a redox active ROS scavenger or generator depending on state), iron oxides (Fe₃O₄ and/or Fe₂O₃) (which can act synergistically with magnetic targeting), manganese dioxide (MnO₂) (which is known to be responsive to redox microenvironments), or any combination thereof.

In still another aspect, the nanoparticles can be polymeric nanoparticles. In one aspect, a polymeric nanoparticle can encapsulate other materials including, but not limited to, metal salts, photosensitizers, or radiocatalysts. In another aspect, polymeric nanoparticles can serve as functional carriers. In still another aspect, polymeric nanoparticles can be functionalized with specific chemical groups to enhance catalytic surfaces. Examples of suitable polymeric nanoparticles include, but are not limited to poly(lactic-co-glycolic acid) (PLGA), polyethylene glycol (PEG) coated systems for improved circulation, chitosan-based nanoparticles, and conductive polymers including, but not limited to, polypyrrole or polyaniline polymers. Further in this aspect, conductive polymers may be useful for charge-transfer purposes in ROS catalysis. In some aspects, the polymers can degrade when exposed to X-rays, causing the release of any payload during irradiation. In some aspects, doping the loaded nanoparticles can enhance the release of payload.

In another aspect, the nanoparticles can be composite nanoparticles. In an aspect, a composite nanoparticle brings together a high atomic number metal with a catalytic surface for generating ROS. These can include, but are not limited to, core-shell structures (e.g. gold core with TiO₂ shell), hybrid systems (gadolinium-doped ZnO or Fe₃O₄—TiO₂ composites), liposome-based nanocarriers loaded with radiolytic catalysts, or any combination thereof.

In some aspects, nanoparticles from more than one class above can be used together. For example, metal and metal oxide nanoparticles can be used together, or polymeric nanoparticles can be used with metal oxide nanoparticles, and so forth.

### Nanoparticle Administration

In one aspect, nanoparticles can be administered in any one of several ways. In one aspect, the nanoparticles can be administered by intravenous injection. Further in this aspect, this may be especially useful when systemic delivery with targeting ligands is desired. Still further in this aspect, the targeting ligands can be molecules such as, for example, transferrin, antibodies, or the like. In another aspect, the nanoparticles can be administered intranasally. Without wishing to be bound by theory, intranasal administration offers the potential for direct access of the nanoparticles to brain tissue. In another aspect, nanoparticles can be delivered by localized injection into CNS targets including, but not limited to, the striatum, the ventral tegmental area (VTA), or spinal cord for regional concentration. In one aspect, convection-enhanced delivery (CED) can be used to deliver the nanoparticles. Further in this aspect, CED may allow the nanoparticles to bypass the blood brain barrier with precision. In any of these aspects, the nanoparticles can be encapsulated in hydrogel or implantable matrix for sustained release.

### Method of Treating Diseases and Disorders

In one aspect, the disclosed method can be used to treat a disease or disorder in a subject. In some aspects, the disease can be a neurological disorder such as, for example, spinal cord injury, amyotrophic lateral sclerosis (ALS), or Parkinson's disease. In still another aspect, the method can be used to modulate a symptom of a disease or disorder in a subject such as, for example, micturition dysfunction resulting from spinal cord injury, neurogenic bladder, or another cause of lower urinary tract dysfunction. In one aspect, viral transfection for treatment of these diseases and disorders can be targeted to the substantia nigra. In another aspect, the method can trigger measurable dopamine release. In still another aspect, the method can provide precise stimulation for clinical applications.

### Method for Treating Brain Cancer in a Subject

Disclosed herein is a method for treating brain cancer in a subject, the method including at least the steps of virally transfecting one or more brain cancer cells in the subject with a gene encoding dTRPA1(A)10b, thereby causing dTRPA1(A)10b channels to be expressed in the one or more brain cancer cells, and delivering X-ray radiation to the one or more brain cancer cells, wherein the X-ray radiation generates H₂O₂ via radiolysis of water and wherein the H₂O₂ activates the dTRPA1(A)10b channels; thereby increasing intercellular calcium and selectively inducing apoptosis. In some aspects, the disclosed method can be adapted to other receptors to achieve similar effects. In a further aspect, the receptors can be dTRPA1(A)10b analogues, such as, for example, mosquito-derived dTRPA1, other heat independent receptors, and/or receptors present on non-neuronal cell types. In still another aspect, the receptors can be natural receptors or can be sequence-modified receptors introduced to cells through viral transfection or other methods. In one aspect, the sequence-modified receptors can be modified to incorporate temperature sensitive properties. In one aspect, the viral transfection can be lentiviral transfection. However, other transfection methods are also contemplated and should be considered disclosed.

In another aspect, and without wishing to be bound by theory, glioblastoma and related cancers possess naturally elevated intrinsic reactive oxygen species (ROS) levels that can be harnessed as part of the disclosed method. In another aspect, the disclosed method is selective for cancer cells due, in part, to their intrinsically high ROS levels, and causes few or no side effects, making the method more tolerable for patients.

In an aspect, amounts of radiation useful in the disclosed method can vary but, in some cases, can be from about 1 mGy to about 500 mGy, or from about 10 mGy to about 250 mGy, or from about 100 mGy to about 150 mGy, or can be about 1, 5, 10, 25, 50, 100, 150, 200, 250, 300, 350, 400, 450, or about 500 mGy, or a combination of any of the foregoing values, or a range encompassing any of the foregoing values. In some aspects, the method can produce about 70 nM H₂O₂ per Gy of X-ray radiation delivered. In one example, treatment of brain cancer may require a higher dose of radiation and/or a longer exposure time since apoptosis of cancer cells is a desired end goal.

In another aspect, X-ray radiation exposure time can be from about 1 ms to about 25 s, from about 1 ms to about 5 s, or from about 5 ms to about 1 s, or from about 100 ms to about 500 ms, or can be about 1 ms, 50 ms, 100 ms, 500 ms, 1 s, 1.5 s, 2 s, 2.5 s, 3 s, 3.5 s, 4 s, 4.5 s, or about 5 s, or a combination of any of the foregoing values, or a range encompassing any of the foregoing values. In still another aspect, the X-ray radiation can be delivered at a frequency or dose rate of from about 0.1 Hz to about 100 Hz, or from about 0.5 Hz to about 50 Hz, or from about 1 Hz to about 10 Hz, or at about 0.1, 0.5, 1, 5, 10, 20, 30, 40, 45, 50, 55, 60, 65, 70, 75, 80, 85, 90, 95, or about 100 Hz, or a combination of any of the foregoing values, or a range encompassing any of the foregoing values. In some aspects, as with brain cancer, the radiation exposure time and dose can be higher or lower depending on the severity of the disease or symptom being treated.

In any of these aspects, the hydrogen peroxide induces an increased level of intracellular calcium ions compared to the level of intracellular calcium ions prior to performing the method. In a further aspect, the hydrogen peroxide can stimulate apoptosis in brain cancer cells.

The disclosed method can be used in medical treatment or for research purposes. In an aspect, the subject can be a human, mouse, rat, guinea pig, hamster, rabbit, cat, dog, cattle, horse, swine, goat, sheep, or non-human primate with brain cancer cells that have been virally transduced with dTRPA1(A)10b or expressing dTRPA1(A)10b by genetic approaches.

In any of these aspects, nanoparticles can be used as described above to enhance effectiveness of the method.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a symptom,” “a neurotransmitter,” or “a neuron,” includes, but is not limited to, mixtures or combinations of two or more such symptoms, neurotransmitters, or neurons, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of X-ray radiation refers to an amount that is sufficient to achieve the desired effect, e.g. achieving the desired level of H₂O₂ production useful for activating dTRPA1(A)10b, whether native to a cell or transfected virally to a cell. The specific level of X-ray radiation required as an effective amount will depend upon a variety of factors including the number and location of neurons being targeted, disease or condition being treated, and any concurrent therapies being administered.

As used herein, the terms “optional” or “optionally” means that the subsequently described event or circumstance can or cannot occur, and that the description includes instances where said event or circumstance occurs and instances where it does not.

“Neuromodulation” refers to a process of altering nerve (neuronal) activity through delivery of a stimulus. The stimulus can be targeted to a specific set of neurons or nervous tissue. The stimulus can be electrical stimulation, a chemical agent, or in an embodiment of the present disclosure, delivery of targeted X-ray radiation to cells capable of producing H₂O₂ via radiolysis of water.

In an aspect, “TrpA1” refers to transient receptor potential cation channel A1. In a further aspect, this protein, once activated, allows calcium entry into the neuron/cell. The TrpA1 Drosophila gene has several splice variants. “dTRPA1(A)10b” is a temperature (thermos) “insensitive” splice variant of the Drosophila gene that, is highly sensitive to hydrogen peroxide and when treated with radiation, is activated.

“Transfection” as used herein refers to the process of introducing nucleic acids into eukaryotic cells. In one aspect, “viral transfection” is when a gene for delivery to a cell is packaged into a viral particle that is incapable of replicating the virus but, through the use of viral infection mechanisms, can introduce the packaged nucleic acid into a cell. In an aspect, viral transfection can be used herein to deliver dTRPA1(A)10b to mammalian neurons.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as Parkinson's disease or brain cancer. The effect can be therapeutic in terms of a partial or complete cure of a disease, condition, symptom or adverse effect attributed to the disease, disorder, or condition. The term “treatment” as used herein can include any treatment of Parkinson's disease, brain cancer, spinal cord injury, or the like, in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for neuromodulation in a subject, the method comprising:
transfecting one or more neurons in the subject with a gene encoding a TRPA1, thereby causing TRPA1 channels to be expressed in the one or more neurons;
delivering X-ray radiation to the one or more neurons;
wherein the X-ray radiation generates H₂O₂ via radiolysis of water and wherein the H₂O₂ activates the TRPA1 channels; thereby increasing intercellular calcium and releasing one or more neurotransmitters.

Aspect 2. The method of aspect 1, wherein the TRPA1 comprises dTRPA1(A)10b.

Aspect 3. The method of aspect 1 or 2, wherein the TRPA1 comprises a dTRPA1(A)10b edited in exon 12 to induce thermal responsivity.

Aspect 4. The method of aspect 1, wherein the TRPA1 comprises a mosquito TRPA1.

Aspect 5. The method of any one of aspects 1-4, wherein transfecting is accomplished with a virus.

Aspect 6. The method of aspect 5, wherein the virus is a lentivirus.

Aspect 7. The method of any one of aspects 1-6, wherein from about 1 mGy to about 500 mGy of X-ray radiation are delivered to the subject

Aspect 8. The method of any one of aspects 1-7, wherein the X-ray radiation is delivered with an exposure time of from about 1 ms to about 25 s.

Aspect 9. The method of any one of aspects 1-8, wherein the X-ray radiation has a frequency of from about 0.1 Hz to about 100 Hz.

Aspect 10. The method of any one of aspects 7-9, wherein performing the method produces about 70 nM H₂O₂/Gy of X-ray radiation delivered.

Aspect 12. The method of any one of aspects 1-10, wherein the subject comprises an isolated plurality of cells, an isolated organ, or an animal subject

Aspect 13. The method of aspect 12, wherein the one or more neurons are mammalian neurons.

Aspect 14. The method of aspect 13, wherein transfecting is accomplished by a physical means or a chemical means.

Aspect 15. The method of aspect 14, wherein the physical means comprises electroporation, microinjection, or biolistics.

Aspect 16. The method of aspect 14, wherein the chemical means comprises Ca2+ phosphate co-precipitation or lipofection.

Aspect 17. The method of aspect 12, wherein the isolated organ comprises a brain.

Aspect 18. The method of aspect 12, wherein the animal subject comprises Drosophila, a human, mouse, rat, guinea pig, hamster, rabbit, cat, dog, cattle, horse, swine, goat, sheep, or non-human primate.

Aspect 19. The method of aspect 13, wherein the one or more neurons comprise neurons in the central nervous system, neurons in the peripheral nervous system, or any combination thereof.

Aspect 20. The method of any one of aspects 1-19, wherein the one or more neurotransmitters comprise dopamine, serotonin, norepinephrine, or any combination thereof.

Aspect 21. The method of any one of aspects 1-20, wherein the method is used to treat a neurological disease or disorder in a subject.

Aspect 22. The method of aspect 21, wherein the neurological disease or disorder comprises spinal cord injury, amyotrophic lateral sclerosis (ALS), or Parkinson's disease.

Aspect 23. The method of any one of aspects 1-20, wherein the method is used to modulate a symptom of a disease or disorder in a subject

Aspect 24. The method of aspect 23, wherein the symptom is micturition dysfunction resulting from spinal cord injury, neurogenic bladder, or another cause of lower urinary tract dysfunction.

Aspect 25. The method of any one of aspects 1-24, further comprising administering nanoparticles to the one or more neurons, wherein the nanoparticles enhance generation of one or more reactive oxygen species.

Aspect 26. The method of aspect 25, wherein the nanoparticles comprise metal nanoparticles, metal oxide nanoparticles, polymeric nanoparticles, composite nanoparticles, or any combination thereof.

Aspect 27. The method of aspect 26, wherein the metal nanoparticles comprise gold, platinum, bismuth, tungsten, silver, gadolinium, tantalum, or any combination thereof.

Aspect 28. The method of aspect 26, wherein the metal oxide nanoparticles comprise TiO₂, ZnO, CeO₂, Fe₃O₄, Fe₂O₃, MnO₂, or any combination thereof.

Aspect 29. The method of aspect 26, wherein the polymeric nanoparticles comprise poly(lactic-co-glycolic acid), polyethylene glycol, chitosan, polypyrrole, polyaniline, or any combination thereof.

Aspect 30. The method of aspect 29, wherein the polymeric nanoparticles encapsulate at least one additional material.

Aspect 31. The method of aspect 30, wherein the at least one additional material comprises a metal salt, a photosensitizer, a radiocatalyst, or any combination thereof.

Aspect 32. The method of aspect 26, wherein the composite nanoparticles comprise a core-shell structure, a hybrid system, or a liposome-based nanocarrier.

Aspect 33. The method of aspect 32, wherein the core-shell structure comprises a gold core with a TiO₂ shell.

Aspect 34. The method of aspect 32, wherein the hybrid system comprises gadolinium-doped ZnO or a Fe₃O₄—TiO₂ composite.

Aspect 35. The method of any one of aspects 25-34, wherein the nanoparticles are administered by intravenous injection, intranasally, localized injection into a CNS target, or convection-enhanced delivery (CED).

Aspect 36. The method of any one of aspects 25-35, wherein the nanoparticles are delivered in conjunction with one or more targeting ligands.

Aspect 37. The method of aspect 36, wherein the one or more targeting ligands comprise transferrin, antibodies, or any combination thereof.

Aspect 38. A method for treating brain cancer in a subject, the method comprising:
transfecting one or more brain cancer cells in the subject with a gene encoding a TRPA1 channel;
delivering X-ray radiation to the one or more brain cancer cells;
wherein the X-ray radiation generates H₂O₂ via radiolysis of water and wherein the H₂O₂ activates the TRPA1 channel; thereby increasing intercellular calcium and selectively inducing apoptosis.

Aspect 39. The method of aspect 38, wherein the TRPA1 comprises dTRPA1(A)10b.

Aspect 40. The method of aspect 38 or 39, wherein the TRPA1 comprises a dTRPA1(A)10b edited in exon 12 to induce thermal responsivity.

Aspect 41. The method of aspect 38, wherein the TRPA1 comprises a mosquito TRPA1.

Aspect 42. The method of any one of aspects 38-41, wherein transfecting is accomplished with a virus.

Aspect 43. The method of aspect 42, wherein the virus is a lentivirus.

Aspect 44. The method of any one of aspects 38-43, wherein from about 1 mGy to about 500 mGy of X-ray radiation are delivered to the subject.

Aspect 45. The method of any one of aspects 38-44, wherein the X-ray radiation is delivered with an exposure time of from about 1 ms to about 25 s.

Aspect 46. The method of any one of aspects 38-45, wherein the X-ray radiation has a frequency of from about 0.1 Hz to about 100 Hz.

Aspect 47. The method of any one of aspect 44-46, wherein performing the method produces about 70 nM H₂O₂/Gy of X-ray radiation delivered.

Aspect 48. The method of any one of aspects 38-47, wherein the subject comprises a human, mouse, rat, guinea pig, hamster, rabbit, cat, dog, cattle, horse, swine, goat, sheep, or non-human primate.

Aspect 49. The method of any one of aspects 38-48, further comprising administering nanoparticles to the one or more neurons, wherein the nanoparticles enhance generation of one or more reactive oxygen species.

Aspect 50. The method of aspect 49, wherein the nanoparticles comprise metal nanoparticles, metal oxide nanoparticles, polymeric nanoparticles, composite nanoparticles, or any combination thereof.

Aspect 51. The method of aspect 50, wherein the metal nanoparticles comprise gold, platinum, bismuth, tungsten, silver, gadolinium, tantalum, or any combination thereof.

Aspect 52. The method of aspect 50, wherein the metal oxide nanoparticles comprise TiO₂, ZnO, CeO₂, Fe₃O₄, Fe₂O₃, MnO₂, or any combination thereof.

Aspect 53. The method of aspect 50, wherein the polymeric nanoparticles comprise poly(lactic-co-glycolic acid), polyethylene glycol, chitosan, polypyrrole, polyaniline, or any combination thereof.

Aspect 54. The method of aspect 53, wherein the polymeric nanoparticles encapsulate at least one additional material.

Aspect 55. The method of aspect 54, wherein the at least one additional material comprises a metal salt, a photosensitizer, a radiocatalyst, or any combination thereof.

Aspect 56. The method of aspect 50, wherein the composite nanoparticles comprise a core-shell structure, a hybrid system, or a liposome-based nanocarrier.

Aspect 57. The method of aspect 56, wherein the core-shell structure comprises a gold core with a TiO₂ shell.

Aspect 58. The method of aspect 56, wherein the hybrid system comprises gadolinium-doped ZnO or a Fe₃O₄—TiO₂ composite.

Aspect 59. The method of any one of aspects 49-58, wherein the nanoparticles are administered by intravenous injection, intranasally, localized injection into a CNS target, or convection-enhanced delivery (CED).

Aspect 60. The method of any one of aspects 49-59, wherein the nanoparticles are delivered in conjunction with one or more targeting ligands.

Aspect 61. The method of aspect 60, wherein the one or more targeting ligands comprise transferrin, antibodies, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Radiolytic Control of Cell Activity and Fate to Target Neurological Disorders

Radiolytic neuromodulation is a novel, non-invasive technology platform that modulates the activity of the central and/or peripheral nervous systems (CNS and PNS, respectively) and muscle cells, with temporal and spatial definition as well as regional and cellular specificity.

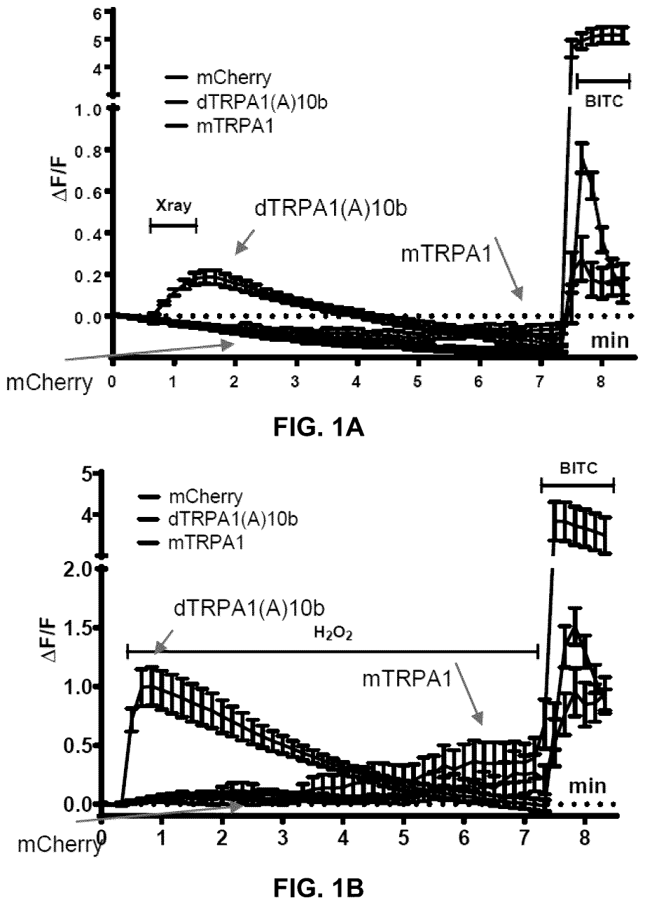

Radiolytic neuromodulation can be used to regulate and repair neuronal function with regional and cellular specificity. Xray can generate low levels of hydrogen peroxide (H₂O₂) through radiolysis of H₂O. Utilizing Xray irradiation, neuronal activity and associated animal behaviors are promoted by activation of the H₂O₂-sensitive transient receptor potential (TRP) ankyrin type-1 (TRPA1) channel found in Drosophila (dTRPA1; see below). Of note is that the mammalian homolog is insensitive to Xray (FIG. 1A). By expressing dTRPA1 in mammalian tissues/cells (e.g. nervous system/neurons), it is possible to control cell activity and programmed death by Xray. This approach allows manipulation of the activity of defined neuronal populations to dictate specific behavior in freely moving animals. This is achieved by virally expressing dTRPA1 in defined neuronal populations and activating them with Xray. Impaired neuronal circuits involved in the pathophysiology of neuropsychiatric and neurological disorders can be corrected by normalizing their function with Xray. Considering that Xray irradiation is a non-invasive technique, with controlled stimulus intensity and high spatial resolution, the development of radiolytic neuromodulation is fundamental for therapeutic advancement.

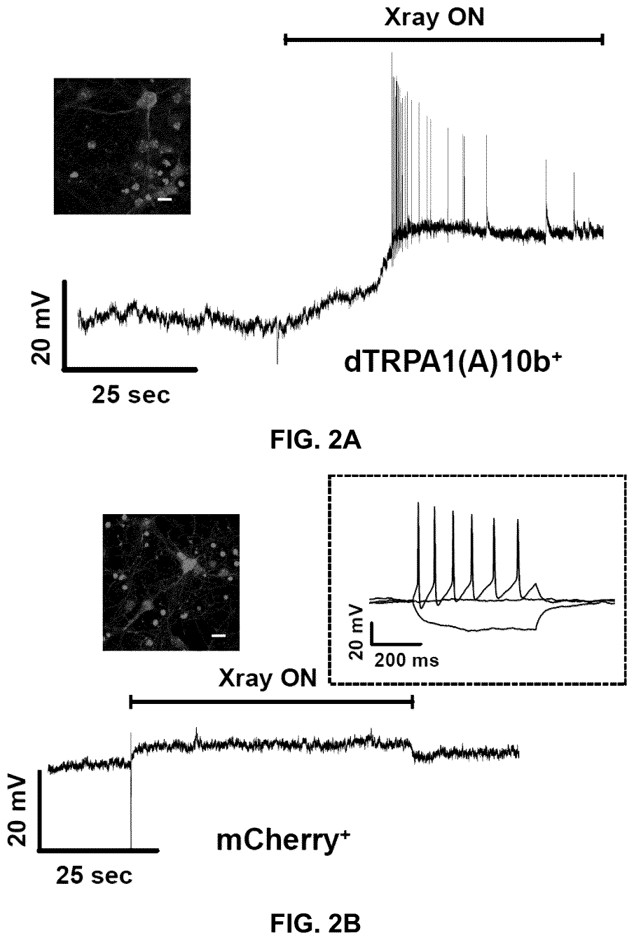

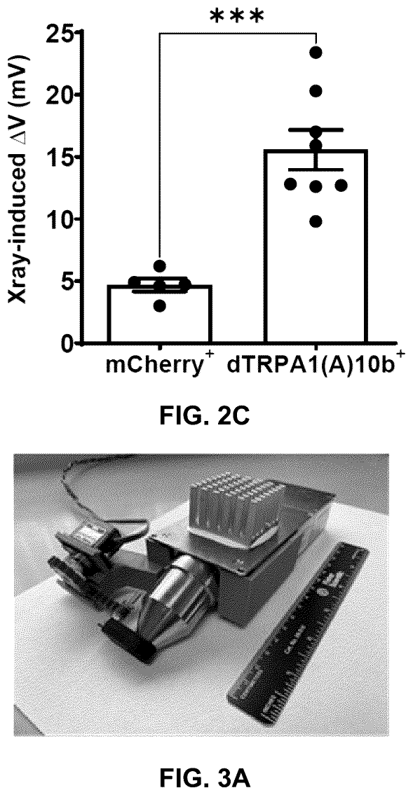

In cells expressing dTRPA1, Xray stimulates neuronal activation by membrane depolarization, neurotransmitter release, as well as an increase in intracellular Ca²⁺. The dTRPA1 channel is an evolutionarily conserved channel that acts as a detector of reactive oxygen species and is permeable to Na⁺ and Ca²⁺. Indeed, H₂O₂ activates the dTRPA1 channel with high affinity. Notably, the dTRPA1(A)10b isoform is highly sensitive to H₂O₂ and is not a direct heat sensor and thus, allows the translation of Drosophila-related discoveries to warm-blooded mammals. In contrast to TRPA1(A)10b, the mammalian homolog of dTRPA1, such as the mouse TRPA1 (mTRPA1) has low sensitivity to H₂O₂. Of note is that irradiation of physiological (aqueous) solutions with Xray rapidly and acutely produces H₂O₂. Thus, it was hypothesized that, in mammalian cell preparations expressing dTRPA1(A)10b, Xray would cause transient increases in intracellular Ca²⁺, resulting in neuronal activation, as well as neurotransmitter release. In FIGS. 1A-1B, it is shown that stimulation of HEK293 cells expressing dTRPA1(A)10b with either Xray (FIG. 1A) or H₂O₂ (FIG. 1B) promotes an acute increase in intracellular Ca²⁺. In contrast, this increase is not observed in cells expressing either mTRPA1 or mCherry control (FIGS. 1A-1B). Finally, benzyl isothiocyanate (BITC), a TRPA1 channel agonist, increases intracellular Ca²⁺ in both TRPA1(A)10b and mTRPA1 expressing cells. Notably, Tempol, a membrane-permeable reactive oxygen species (ROS; e.g. H₂O₂) scavenger, blocked Xray-induced Ca²⁺ increases, demonstrating that Xray is required to produce ROS species in order to increase intracellular Ca²⁺. In order to demonstrate the translational importance of radiolytic neuromodulation to target neuronal activity and neurological disorders, dTRPA1(A)10b was expressed in cortical neurons. FIG. 2A shows that in cortical neurons expressing dTRPA1(A)10b (picture), Xray stimulation (0.25 nGy/cm²/sec) causes cell membrane depolarization and action potential firing. Notably, in neurons expressing mCherry only (FIG. 2B, picture), Xray stimulation failed to cause a significant membrane depolarization and action potential firing (FIG. 2B). To validate the integrity of neuronal excitability of these neurons, action potential firing was stimulated (FIG. 2B, inset) with incremental depolarizing current-injections of 500 ms (50 pA increments of three steps). FIG. 2C shows that Xray stimulation causes a significant increase in membrane potential depolarization in dTRPA1(A)10b expressing neurons with respect to mCherry neurons (control). Consistent with the ability of Xray to cause neuronal activation, in Drosophila brains, which endogenously express dTRPA1(A)10b, Xray (0.1 μGy/cm²/sec) caused TTX-sensitive, dTRPA1(A)-dependent vesicular release of neurotransmitter.

Radiolytic neuromodulation is the first non-invasive approach capable of inducing neurotransmitter release with controlled stimulus intensity. Controlling levels of activation of specific brain regions/neurocircuits will enable discovery of specific behaviors associated with this activation. Specific neurocircuits in the CNS can be normalized for potential therapeutic option. For example, in the CNS, Xray can be used with intersectional genetics to correct motor deficits in mouse models of Parkinson's Disease by activating dopaminergic nodes. The present technology can also be used for regulation of the lower urinary tract symptoms (LUTS) and function in Parkinson's patients. Neuromodulation techniques in clinical use and other experimental techniques (i.e. optogenetics) to manage LUTS require surgical device implantation. Radiolytic neuromodulation can be adopted to regulate micturition in a mouse model with complete loss of LUT function (e.g. spinal cord injury). A focused Xray system with Pulse Width Modulation (PWM) intensity control can be developed, which will allow for precise millimetric spatial targeting of the CNS and PNS with high temporal resolution and intensity control. The present technology can provide insights into how changes in the frequency and strength of activation of specific neurocircuits cause specific behaviors. The present technology can further be used to develop a novel genetic-based, cell death-promoting, cancer therapeutic. Intracellular levels of ROS are tonically elevated in multiple types of cancer (e.g. glioblastoma). The ROS-sensitive Ca²⁺ permeable dTRPA1(A)10b channel can be expressed to achieve sustained, not transient, elevation of intracellular Ca²⁺, initiating apoptosis specifically in cancer cells. Cell death will further be promoted by elevating ROS levels with radiotherapy, thereby preventing development of therapeutic resistance.

In the future, radiolytic neuromodulation has the potential to be broadly applied to any tissue to gain control of cell activity and fate to target muscle control, hormonal regulation, and promote programmed death in dysregulated cells with high temporal and spatial precision.

### Example 2: Xray as Stimulus for Controlling Neurotransmitter Release With Regional/Local Specificity

Herein it is demonstrated that Xrs stimulate an increase in intracellular Ca²⁺ as well as neurotransmitter release and associated behaviors in biological preparations expressing the Drosophila transient receptor potential (dTRP) ankyrin type-1 (dTRPA1) channel. The dTRPA1 channel is an evolutionarily conserved detector of reactive oxygen species. Indeed, hydrogen peroxide (H₂O₂) activates the dTRPA1 channel with high affinity. The dTRPA1 locus has two promoters (A and B) that drive expression of at least five transcripts. The dTRPA1(A)10a and dTRPA1(A)10b isoforms are derived from the A promoter and are highly sensitive to H₂O₂. However, the dTRPA1(A)10b isoform is not a direct heat sensor and thus, distinct from dTRPA1(A)10a. Importantly, irradiation of physiological (aqueous) solutions with Xrs rapidly produces H₂O₂, strongly suggesting that the increase in intracellular Ca²⁺ and neurotransmitter release promoted by Xrs in preparations expressing dTRPA1(A)10b are events mediated through Xrs-induced H₂O₂ synthesis and consequently, activation of dTRPA1 channels. Xrs stimulation does not cause neurotransmitter release in murine brain preparations, a phenomenon that is attributed to the low sensitivity of the mouse TRPA1 (mTRPA1) channel to H₂O₂. Thus, viral expression of dTRPA1(A)10b in genetically defined neurons in murine animal models can allow Xrs to control their activity and associated behaviors.

Xrs, through activation of dTRPA1(A)10b, has been demonstrated to induce increases in intracellular Ca²⁺, neurotransmitter release, and behaviors, using cells, neuronal lines, and Drosophila as model systems expressing dTRPA1(A)10b. This approach is defined herein as Xrs genetics. Xrs genetics can be translated to warm-blooded mammals by virally expressing the dTRPA1(A)10b in genetically defined neurons. This allows Xrs to activate these neuronal populations and promote associated behaviors. dTRPA1(A)10b will be used instead of dTRPA1(A)10a, since the former is not a direct heat sensor and thus, allows the translation of this technology to warm-blooded mammals.

Preliminary data demonstrates that Xrs stimulates neurotransmitter release, as well as an increase in intracellular Ca²⁺. These events are mediated, at least in part, by the Drosophila transient receptor potential (dTRP) ankyrin type-1 (dTRPA1) channel, an evolutionarily conserved detector of irritant chemicals and reactive oxygen species. Indeed, hydrogen peroxide (H₂O₂) activates the dTRPA1 channel with high affinity. The dTRPA1 locus has two promoters (A and B) that drive expression of at least five transcripts. The dTRPA1(A)10a and dTRPA1(A)10b isoforms are derived from the A promoter, and are highly sensitive to H₂O₂. However, dTRPA1(A)10b is not a direct heat sensor and thus, distinct from dTRPA1(A)10a. Of note is that irradiation of physiological (aqueous) solutions with Xrs rapidly produces H₂O₂. Herein, it is demonstrated that Xrs induces an increase in intracellular Ca²⁺ in cells expressing the dTRPA1(A)10b, as well as synaptic neurotransmitter release in Drosophila brains. This data also strongly suggests that these events are mediated through Xrs-induced H₂O₂ synthesis and, as a consequence, activation of neuronal dTRPA1 channels. Significantly, Xrs stimulation (at the doses used herein) does not cause neurotransmitter release in murine brain preparations, a phenomenon attributed to the low sensitivity of the mouse TRPA1 (mTRPA1) channel to H₂O₂. Therefore, expression of dTRPA1(A)10b in select murine brain regions/neuronal populations can create a sensor for Xrs to allow activation of specific neuronal populations. dTRPA1(A)10b is used instead of dTRPA1(A)10a, since the former is not a direct heat sensor and thus, allows the translation of these discoveries to warm-blooded mammals.

Herein, it is mechanistically described how Xrs, through activation of dTRPA1(A)10b, induce increases in intracellular Ca²⁺, neurotransmitter release, and behaviors, using cell lines, neuronal lines, and Drosophila as model systems expressing endogenous dTRPA1(A)10b. Flies are used as a model system to systematically examine the molecular hypotheses in a time- and cost-effective manner. These experiments will provide the foundation to support stimulation of neurotransmitter release and behaviors in mice with Xrs. This will be achieved by viral expression of the dTRPA1(A)10b channel (more sensitive to H₂O₂ than mTRPA1, as well as being temperature insensitive) in specific regions and/or neuronal subtypes of the mouse brain. Since the Xrs dose rate (Gray (Gy)/s) will determine the rate of H₂O₂ production, and consequently, the level dTRPA1 channel stimulation, Xrs can also afford an opportunity to control the strength of dTRPA1(A)10b-mediated neuronal activation through fine tuning of Xrs dose rate and thereby, the rate of H₂O₂ synthesis. This presents an opportunity to induce regional/neuronal-specific neurotransmitter release in a freely moving mouse with a non-invasive technique, and to associate this release with specific behaviors.

To facilitate the development of Xrs genetics, an Xrs source was used to target cell preparations, neurons, isolated brains of Drosophila, as well as live flies, to monitor changes in intracellular Ca²⁺, neurotransmitter release and associated behaviors promoted by Xrs stimulation. The Drosophila dopamine (DA) system was used as proof-of-concept of neurotransmitter release. For brain stimulation, the Xrs source is mounted on an electrophysiological rig to measure increases in Ca²⁺ by confocal imaging or DA release by amperometry, a technique that measures DA levels by oxidation. It was demonstrated that low doses of Xrs promote both an increase in intracellular Ca²⁺ in cells expressing dTRPA1(A)10b, as well as DA release in isolated fly brains; both these events are dTRPA1(A)10b-dependent. It was further demonstrated that perfusion of H₂O₂ also causes Ca²⁺ increases and DA release. These data afford the opportunity to establish Xrs as a technology to induce neurotransmitter release in specific neuronal populations in an intact murine brain.

In flies, DA controls many behaviors, including locomotion. To begin to phenotypically translate these discoveries in cells and isolated brains, herein it is shown that in Drosophila, Xrs stimulation significantly increases locomotion, as does exposure to H₂O₂. These data suggest that Xrs stimulation of DA release is associated with DA-dependent behaviors.

Herein are determined the energetic modalities of Xrs-induced increases in intracellular Ca²⁺, neurotransmitter release, and associated behaviors, taking advantage of flies as an animal model.

Preliminary data demonstrates that Xrs stimulate increases in intracellular Ca²⁺ and neurotransmitter release, in biological preparations expressing the Drosophila transient receptor potential (dTR P) ankyrin type-1 (dTRPA1) channel.

The transient receptor potential (TRP) channels: are variably selective cation channels that consist of six transmembrane spanning domains and are permeable to Ca²⁺ and Na⁺ ions. They are polymodal cellular sensors involved in a wide variety of cellular processes, mainly by changing membrane voltage and increasing intracellular Ca²⁺. Conserved through evolution, they are expressed in most tissues and cell-types. Based on amino acid homology, the TRP channel superfamily is typically classified into ten related subfamilies: TRPA1, TRPV, TRPVL, TRPC, TRPM, TRPS, TRPN, TRPY/TRPF PKD2s, and TRPML. They participate in and regulate many physiological processes, including pain, thermoregulation, as well as sensitivity to free radicals.

In Drosophila, the dTRPA1 channel is an evolutionarily conserved detector of irritant chemicals and reactive oxygen species (ROS). Indeed, the ROS specie hydrogen peroxide (H₂O₂) activates the dTRPA1 channel with high affinity. It has been shown that short wavelength ultraviolet (UV) and blue light can trigger ROS and H₂O₂ production. In many insects, including Drosophila, rhythmic short wavelength light avoidance is crucial for avoiding heat, low humidity, and peak ultraviolet (UV) radiation at midday and thus, minimizes a range of hazards from desiccation at organism level to DNA damage at molecular level. Notably, UV light activates dTRPA1 via photochemical production of H₂O₂. The dTRPA1 locus has two promoters (A and B) that drive expression of at least five transcripts. The dTRPA1(A)10a and dTRPA1(A)10b isoforms are derived from the A promoter and are highly sensitive to H₂O₂. However, in contrast to dTRPA1(A)10a, dTRPA1(A)10b is not a direct heat sensor and thus distinct from dTRPA1(A)10a. These data underscore the opportunity to utilize rapid H₂O₂ synthesis as a tool to regulate dTRPA1(A)10b function and, as a consequence, activation of dTRPA1(A)10b expressing neurons.

All air-living organisms express enzymes that limit oxidative stress by neutralizing reactive oxygen species, including H₂O₂. In eukaryotes, H₂O₂ plays an important role in the regulation of a variety of biological processes. The use of a cytotoxic chemical as a signaling molecule has obvious potential risks, so it is no surprise that the generation of H₂O₂ is tightly regulated. In Drosophila, H₂O₂ regulates UV sensitivity, light-induced feeding inhibition, as well as motor activity.

Xrs are known to generate ROS in biological systems. Xrs can generate oxygen radicals by interacting with any available source of oxygen, including water. The predominant effect of Xrs on biological samples is the radiolysis of water to produce hydroxyl radicals and hydrated electrons. At biologically relevant pH, these products react with the surrounding water and other molecules to yield H₂O₂ within 10⁻⁶ seconds. In fact, irradiation of biofluids with Xrs produces H₂O₂ (˜70 nM H₂O₂/Gray). Thus, due to the relative abundance of water in brain, low doses of Xrs can be used to stimulate dTRPA1(A) by harnessing H₂O₂ production. Since the Xrs dose rate (Gray (Gy)/s) will determine the rate of H₂O₂ production, and consequently, the level dTRPA1 channel stimulation4, Xrs might also afford an opportunity to control the strength of dTRPA1(A)10b-mediated neuronal activation by fine tuning Xrs dose rate and thereby, the rate of H₂O₂ synthesis.

Drosophila as an experimental model of whether and how Xrs stimulates neurotransmitter release and the behavioral consequences of the same, allows for conduction of experiments in a time- and cost-effective manner: Most genes involved in neurotransmitter synthesis, transport, secretion, and signaling are conserved among species. Thus, Drosophila is an appropriate genetic model organism to study whether and how dTRPA1(A)10b mediates Xrs stimulation of neurotransmission, ex vivo and in vivo. Using flies, the behavioral consequences of Xrs stimulation of neurotransmitter release can be examined, as well as whether pharmacological/molecular/genetic manipulations inhibiting this release also inhibit the associated behaviors. Preliminary data implicate Xrs as a technology able to stimulate neurotransmitter release and associated behaviors in Drosophila. Considering that it is possible to record dopamine (DA) release in isolated Drosophila brain by amperometry, vesicular DA release can be monitored at first to determine the ability of Xrs to induce neurotransmitter release in tissues expressing the dTRPA1.

The use of Drosophila is also justified since the expression/silencing of specific genes (e.g. dTRPA1 RNAi) can be easily manipulated specifically in DA neurons to determine their importance in vesicular DA release as well as DA associated behaviors (i.e. locomotion, grooming). Several RNAi lines directed against all isoforms of dTRPA1 exist, while TH-Gal4 flies driving mCherry RNAi in DA neurons can be used as a control.

Preliminary data demonstrate that Xrs irradiation increases intracellular Ca²⁺ in biological preparations expressing dTRPA1(A)10b only. This suggests that this Xrs phenomenon is mediated by activation of dTRPA1(A)10b through Xrs-induced H₂O₂ synthesis. This also demonstrates that Xrs causes DA release in isolated Drosophila brains in a TRPA1 dependent manner. Importantly, these Xrs capabilities are not observed in biological preparations expressing the mouse TRPA1 (mTRPA1), a phenomenon that is attributed to the low sensitivity of mTRPA1 channel to H₂O₂.

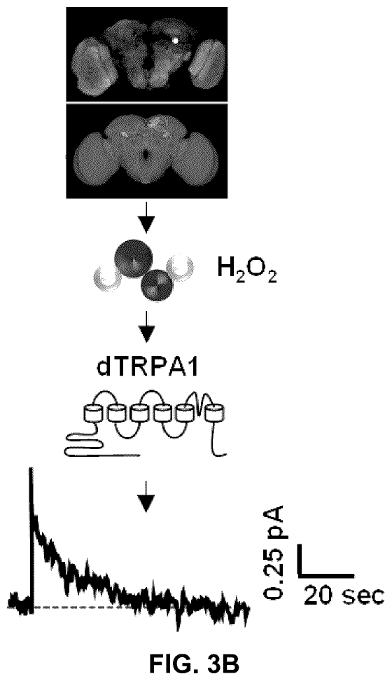

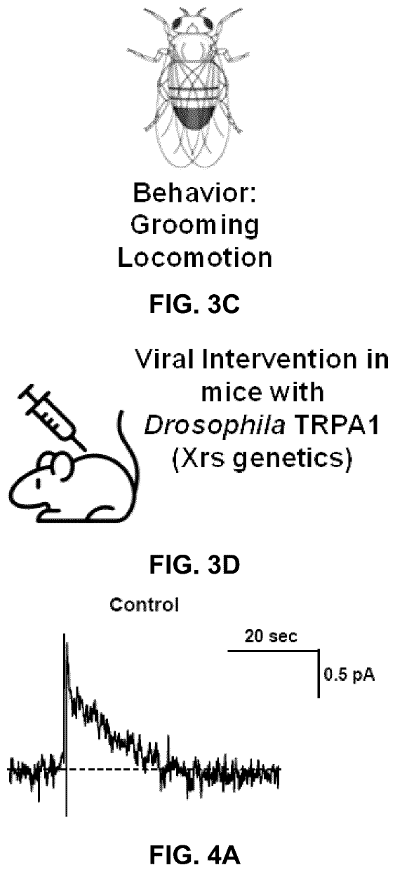

Preliminary data demonstrate that Xrs increases intracellular Ca²⁺ in HEK 293 cells expressing dTRPA1(A)10b (dTRPA1(A)10b cells). In contrast, mTRPA1 cells do not display this increase. Moreover, only in dTRPA1(A)10b cells was this increase stimulated by H₂O₂ exposure. Indeed, the dTRPA1 channels have high sensitivity to H₂O₂, a byproduct of irradiation of physiological solutions with Xrs. To define the dose dependence and kinetics of Xrs-induced neurotransmitter release and increase in intracellular Ca²⁺ in cells, an Xrs source (FIG. 3A) can be used to irradiate isolated Drosophila brains (FIG. 3B, top) or cells. As proof-of-principle that Xrs causes neurotransmitter release in preparation for expressing dTRPA1(A)10b, Xrs-induced DA release was recorded in isolated fly brain. A carbon fiber electrode (5 μm) is inserted into the fly brain (white circle) in proximity to the pedunculus-medial lobe (PML) of the mushroom body (MB), a brain region enriched in dopaminergic projections from protocerebral posteriolateral dopaminergic cluster neuron 1 (PPL1, yellow; virtual fly brain). It was hypothesized that Xrs irradiation of fly brains causes H₂O₂ synthesis and dTRPA1 channel stimulation (FIG. 3B, middle) and, as a consequence, DA release. Indeed, Xrs stimulation caused DA release recorded with the carbon fiber electrode (FIG. 3B, bottom). Utilizing the UAS/TH-GAL4 system to express RNAi constructs against dTRPA1, it was demonstrated that Xrs-induced DA release in isolated fly brains is mediated, at least in part, by dTRPA1 expressed in Drosophila DA neurons.

Flies of both sexes between days 3-8 of age were used. All procedures and data collection are performed blind to genotype and experimental condition. Further studies were designed using statistical power calculations (GPower 3.1) considering means and standard errors from preliminary data. For example, for brain amperometry (n=number of brains), the calculation rendered a minimum sample size of 6 for each experimental group (power=80%, α=0.05). A maximum attrition of 33% was estimated; thus, 9 fly brains were used per group. Using the same strategy, a sample size of n=10 flies were used per group for grooming.

Release of neurotransmitters by Xrs in murine models lacking endogenous dTRPA1 channels can be induced by viral gene delivery of the dTRPA1(A)10b channel in specific nodes/neuronal populations (FIG. 3D).

Determining the impaired neuronal circuits involved in the pathophysiology of neuropsychiatric disorders and intervening to normalize them is fundamental for therapeutic advancement. Thus, Xrs genetics is useful as a non-invasive tool capable of normalizing specific neurocircuits for potential therapeutic option in humans. Furthermore, Xrs genetics is the first non-invasive approach capable of inducing neurotransmitter release with controlled stimulus intensity by the fine tuning of Xrs dose rate.

Xrs irradiation or H₂O₂ administration induces increases in intracellular Ca²⁺ in dTRPA1(A)10b expressing cells only: In FIGS. 1A-1B, it is shown that stimulations of HEK293 cells expressing dTRP A1(A)10b with either Xrs (FIG. 1A) or H₂O₂ (FIG. 1B) promotes an increase in intracellular Ca²⁺. In contrast, this increase is not observed in cells expressing either mTRPA1 or mCherry control (FIGS. 1A-1B). The Xrs source (FIG. 3A) was mounted to a Zeiss 710 LSM confocal imaging system. Changes in intracellular Ca²⁺ were detected using a Fluo-4 Direct™ calcium assay kit. Fluo-4 epifluorescence changes were computed as (Fi-Fo; ΔF)/Fo, where Fi, is the fluorescence intensity at any frame and F₀, is the resting fluorescence. F₀ was calculated as the averaged fluorescence of the first 3 frames before stimulation. These data demonstrate that both Xrs and H₂O₂ activate only the dTRPA1(A)10b orthologs. Finally, benzyl isothiocyanate (BITC) a TRPA1 channel agonist increases intracellular Ca²⁺ in both TRPA1(A)10b and mTRPA1 expressing cells. A modest increase in Ca²⁺ induced by BITC was also observed in mCherry control, perhaps because TRPV1-2 are expressed in HEK293 and are a target of BITC.

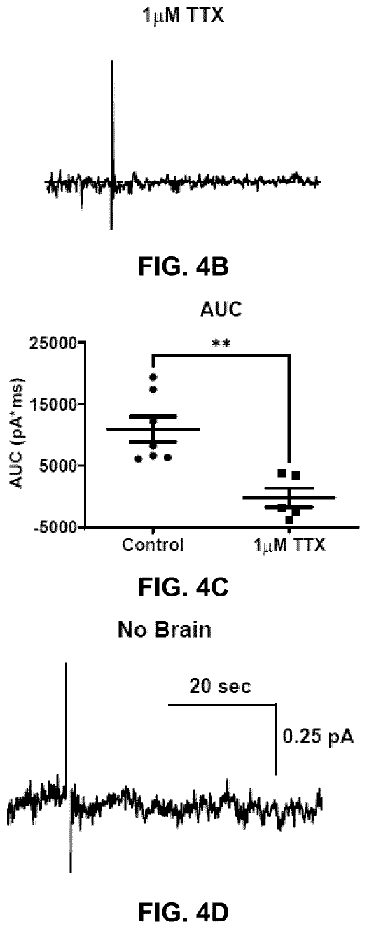

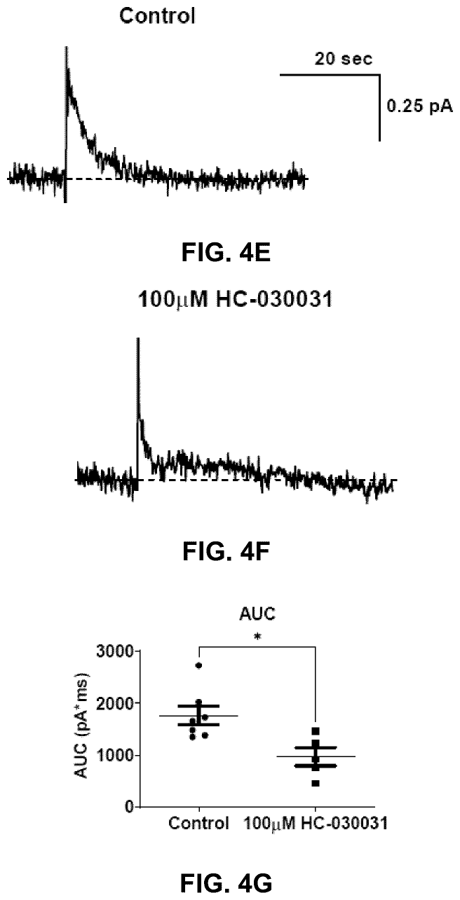

Xrs-stimulated neurotransmitter (i.e. DA) release can be recorded in isolated Drosophila brains by amperometry. The Xrs source (FIG. 3A), is mounted on the amperometric rig in close proximity to the fly brain (2 cm). A carbon fiber electrode (5 μm) is positioned in proximity to the PML of the MB (FIG. 3B, top). To begin to develop Xrs genetics, the synaptic nature of this DA release was first defined. Isolated brains were exposed to either a control solution or a solution containing 1 μM tetrodotoxin (TTX, a Na⁺ channel blocker) (FIGS. 4A-4G). In control conditions, Xrs irradiation of a fly brain promotes DA release (FIG. 4A). However, TTX exposure robustly inhibits this release (FIG. 4B). TTX inhibition of Xrs-induced DA release was quantified by integrating the amperometric current (AUC; pA*msec) (FIG. 4C). The amperometric signal is not an artifact promoted by Xrs stimulation since in the absence of fly brain, Xrs do not affect the amperometric current (FIG. 4D). Next, the involvement of dTRPA1 in this Xrs stimulation of DA release was determined. FIGS. 4E-4F show that the dTRPA1 channel antagonist HC-03003127 inhibits Xrs-stimulated DA release with respect to vehicle. HC-030031 inhibition was quantified calculating the AUC of the amperometric signal (FIG. 4G).

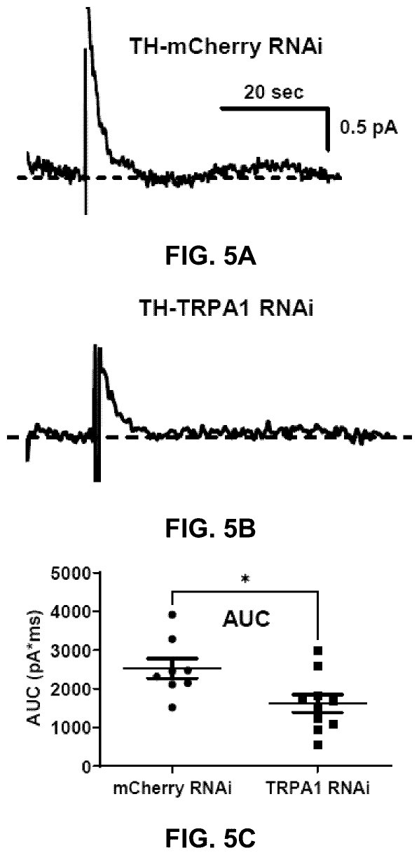

To further characterize the DA-dependent nature of the Xrs-stimulated amperometric signal, either 3-iodotyrosine (3IY; 10 mg/mL) or vehicle was orally administered to flies for 24 hr. 3IY is an inhibitor of DA synthesis used to determine the effects of decreased DA levels in social spacing of Drosophila and other behaviors. The AUC of the amperometric signals show that DA depletion with 3IY significantly reduces the ability of Xrs to stimulate DA release (Student's t-test; *=p<0.013; n=13-15). FIGS. 4A-4G demonstrate that pharmacological inhibition of dTRPA1 significantly inhibits Xrs-induced DA release. To determine whether the dTRPA1 channels expressed in DA neurons support the ability of Xrs to stimulate DA release, the Gal4/UAS system was adopted to generated RNAi against dTRPA1 specifically in DA neurons. This system has two parts: the Gal4 gene, encoding the yeast transcription activator protein Gal4, and the upstream activation sequence (UAS), a minimal promoter region to which Gal4 specifically binds to activate transcription. Flies were developed where the Gal4 expression is driven by the tyrosine hydroxylase (TH) promoter (TH-GAL4), driving the expression of Gal4 specifically in DA neurons (in flies, octopamine, the analog of norepinephrine, does not require TH for synthesis). In these experiments, TH-GAL4 female flies (Bloomington Stock #8848) were crossed with either UAS-TRPA1 RNAi or UAS-mCherry RNAi flies. This crossing results in flies that express RNAi for either TRPA1(A) or mCherry (control) specifically in DA neurons. Brains of flies expressing either TH-mCherry (FIG. 5A) or TH-TRPA1 RNAi (FIG. 5B) were irradiated with Xrs. The AUC of the amperometric signals is significantly reduced in flies expressing TRPA1 RNAi (FIG. 5C).

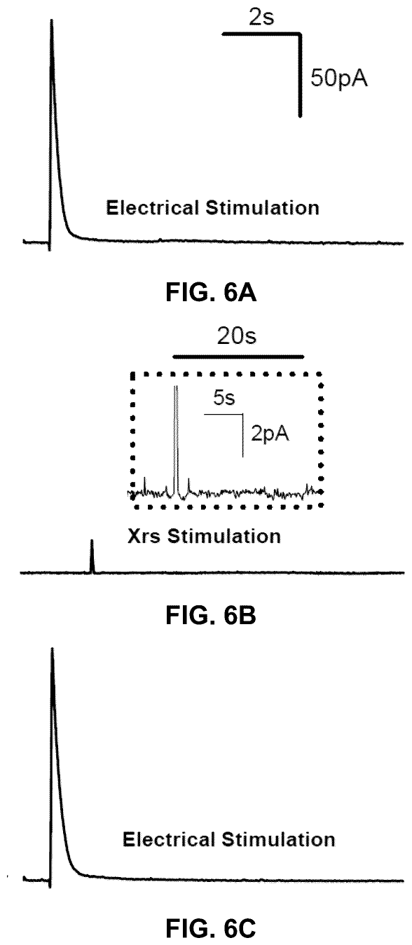

In mice, TRP channels are expressed in a variety of neurons, including DA neurons. However, the sensitivity of mTRPA1 to H₂O₂ is extremely reduced compared to dTRPA1(A)10a-b. It was determined whether a dose of Xrs stimulation (˜0.1 Gy/sec; 250 msec) that caused DA release in fly brains could cause DA release in mouse striatal preparations. DA release was first evoked by electrical stimulation with a bipolar electrode and recorded it by amperometry, as previously described (FIG. 6A). Next, the same striatal slice was stimulated with Xrs. In contrast to the electrical stimulation, Xrs failed to cause DA release (FIG. 6B), probably due to the low sensitivity of the mTRPA1 channels to H₂O₂. Noticeable is a sharp and brief artifact lacking any kinetics in the decay (FIG. 6B, inset). However, a second electrical stimulation of the same striatal slice caused DA release again, indicating that the slice was not DA depleted (FIG. 6C).

Preliminary data show that Xrs induce a robust increase in intracellular Ca²⁺ in cells expressing dTRPA1(A)10b (FIGS. 1A-1B), as well as a DA release in fly brains. This DA release is TTX sensitive, TRPA1 (FIGS. 4A-5C), and DA dependent. The increase in intracellular Ca²⁺ and DA release is not observed in preparations expressing mTRPA1 (FIGS. 1A-1B and 4A-6C) and thus, dTRPA1 dependent. However, the stimulus parameters have not yet been optimized. Initially, a minimal dose of Xrs (0.1 Gy/sec) was used with an exposure time of 250 ms. To develop Xrs genetics, both the energetics and the dynamics of Xrs-stimulated neurotransmitter release must be understood. Precise control of time and energy of Xrs irradiation of cells and fly brains will be employed in order to do so. Preliminary data also support the idea that Xrs-induced DA release is mediated by H₂O₂ synthesis and, consequently, dTRPA1 activation. Combining pharmacological and genetic approaches, this process will be mechanistically defined in Drosophila brains.

It is important to define the energetic (dose) modalities of Xrs stimulation and how they translate to an increase in intracellular Ca²⁺ in dTRPA1(A)10b cells and DA release. This is because the Xrs dose determines the amount of H₂O₂ produced, and therefore, the level of dTRPA1 channel stimulation. Thus, these results represent an opportunity to learn how to control the strength of Ca²⁺ dependent neurotransmitter release through fine-tuning of Xrs dose and therefore, H₂O₂ synthesis. Varying amounts of radiation Xrs (1-500 mGy) and time of exposure (1 ms-5 sec) will be delivered to dTRPA1(A)10b cells and isolated fly brains, as in FIGS. 1A-1B and 4A-4G. The optimal dose of Xrs to increase intracellular Ca²⁺ and to cause DA release as in FIGS. 1A-1B and 4A-4G will be defined. This optimal dose of Xrs will be tested in cells expressing mCherry (control), mTRPA1, or dTRPA1(A)10b, as in FIGS. 1A-1B. Since it is shown that this DA release is TTX sensitive and dTRPA1 dependent (FIGS. 4A-5C), the TTX sensitivity and dTRPA1 dependence will be determined using the optimal Xrs dose rate. The optimal Xrs dose rate will also be used to determine the DA nature of the amperometric signal, either by DA depletion or generating flies where TH-GAL4 drives RNAi against TH (required for DA synthesis) in DA neurons. RNAi targeting mCherry in DA neurons will serve as mock RNAi.

The irradiation of H₂O by Xrs rapidly (10⁻⁶ seconds) yields H₂O₂. This rapid synthesis of H₂O₂ allows the time dependence of H₂O₂-induced increases in intracellular Ca²⁺ and DA release to be probed by changing the time course of Xrs irradiation. The frequency of irradiation can be controlled by using a custom-built lead shutter and a spinning disk chopper with variable apertures mounted between the Xrs source and lens (spinning disk Xrs chopper; Scitec Instruments). This shutter system allows a variety of waveforms, including steps, pulses and sinusoids. The optimal Xrs dose rate will be delivered at multiple frequencies (0.1-100 Hz). The time-dependence, the refractory times, and recovery times of Xrs-induced increases in intracellular Ca²⁺ and DA release will be determined.

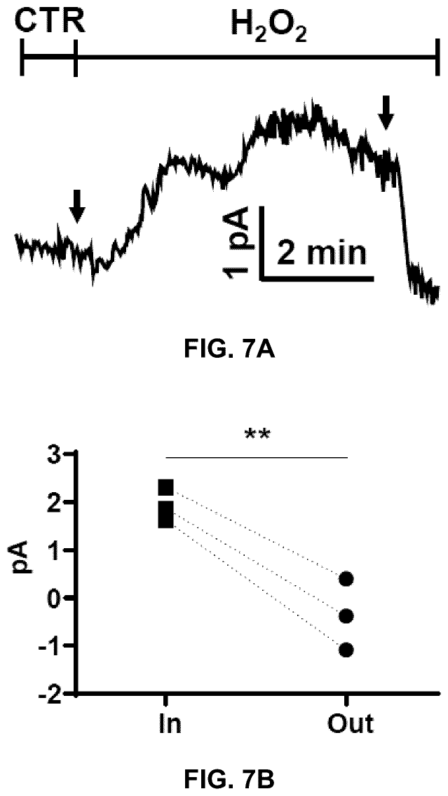

Using genetically encoded reporters for H₂O₂, it was demonstrated that in cells, Xrs exposure causes a rapid increase in H₂O₂, as well as elevation of cytosolic Ca²⁺. It is proposed that Xrs-induces an increase in intracellular calcium in cells expressing TRPA1(A)10b, as well as DA release in fly brains through H₂O₂ synthesis and, as a consequence, TRPA1(A) activation. Consistently, it is shown that H₂O₂ stimulates an increase in intracellular Ca²⁺ in cells expressing TRPA1(A)10b only (FIG. 1B), as well as DA release (FIGS. 7A-7B). Isolated brains of flies (FIGS. 3A-3D) were perfused first with vehicle to record a baseline (FIG. 7A, CTR) and then with 200 μM H₂O₂ (FIG. 7A, red arrow), and DA release recorded with amperometry. After obtaining a stable peak of the amperometric current, the amperometric electrode was pulled out from the brain to determine the background amperometric current in the presence of H₂O₂ (FIG. 7A, green arrow). In FIG. 7B, the peak amperometric currents recorded in the presence of H₂O₂ with the electrode in (red squares) or out (green circles) of the brain are shown. Dose-response curves for H₂O₂-induced increase in intracellular Ca²⁺ (see FIGS. 1A-1B) and DA release (FIGS. 7A-7B) will be generated by perfusing either TRPA1(A)10b cells, mTRPA1 cells, or Drosophila brains with different concentrations of H₂O₂ (0.1-200 μM). In addition to H₂O₂, other agonists of dTRPA1 channels, such as benzyl isothiocyanate (BITC; 1-100 μM) can be used. Next, these preparations (cells or brains) will be perfused with dTRPA1 channels antagonists, such as HC-030031 (1-100 μM), AP18 (1-10 μM), and A-967079 (0.05-0.5 μM), either alone or in combination with H2O2/BITC. In these experiments, the concentration of either H₂O₂ or BITC used (i.e. EC₅₀) will be determined by the results of the dose-response experiments. The experiments involving H2O2/BITC will be repeated in brains from flies with either dTRPA1 RNAi or mock RNAi specifically targeted to DA neurons.

Preliminary data (FIGS. 4A-4G) demonstrates that pharmacological inhibition of dTRPA1 significantly inhibits Xrs-induced DA release. Here, the role of TRPA1 in this Xrs phenomenon will be further described. Drosophila brains will be perfused with either vehicle or the appropriate concentrations of each dTRPA1 channel antagonists, and DA release stimulated with the optimal Xrs dose rate. Furthermore, in FIGS. 5A-5C it is shown that the ability of Xrs to stimulate DA release is supported by the dTRPA1 channel expressed in DA neurons. In these experiments, either RNAi against TRPA1 or mCherry (mock) were generated specifically in DA neurons. These experiments will be repeated with the optimal Xrs dose rate. Other RNAi lines targeting dTRPA1 crossed with TH-GAL4 flies can also be used.

The role played by dTRPA1 channels in Xrs stimulation of DA release, as well as the involvement of H₂O₂, can be described. Since dTRPA1 RNAi, specifically in DA neurons, as well as HC-030031, reduces the ability of Xrs to cause DA release, it is expected that both dTRPA1 RNAi and blockade of dTRPA1 also inhibit the ability of H₂O₂/BITC to cause DA release. However, dTRPA1 RNAi, specifically in DA neurons, did not completely inhibit Xrs-induced DA release. Thus, flies with the pan-neuronal Gal4 driver nSyb (Synaptobrevin)-GAL4 can be crossed with UAS-dTRPA1 RNAi flies, to express the dTRPA1 RNAi in all neurons. This will reveal if other neurocircuits expressing dTRPA1 channels by regulating DA neuronal nodes also participate in Xrs stimulation of DA release.

The abilities of an animal to ambulate in order to feed (locomotion) and/or to be noteworthy (grooming) are behaviors that are essential for the reproductive fitness of animals, including flies. In flies, these behaviors are regulated by DA signaling. It will be determined whether Xrs-induced DA release regulates locomotion and grooming, and whether this regulation requires dTRPA1(A) channels. The phenotypic importance of Xrs-induced DA release will be defined.

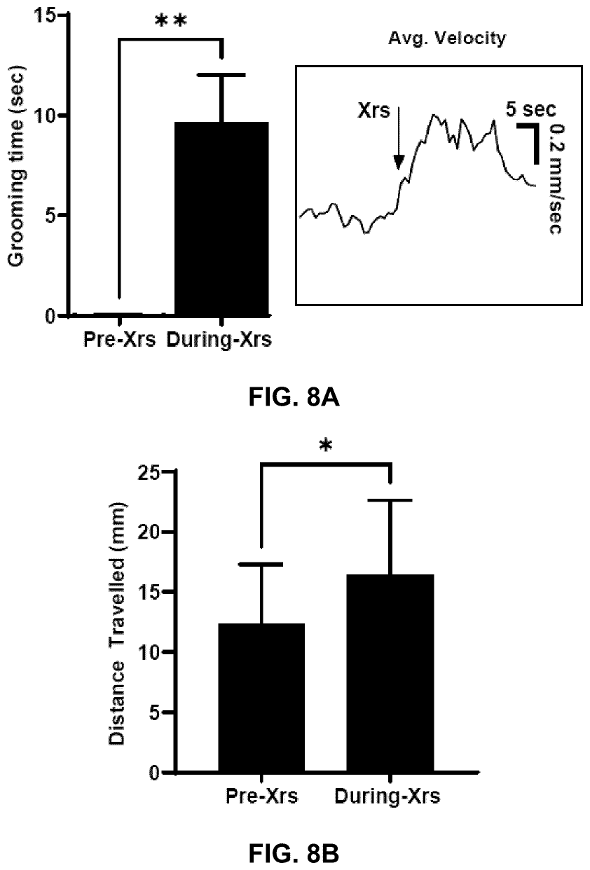

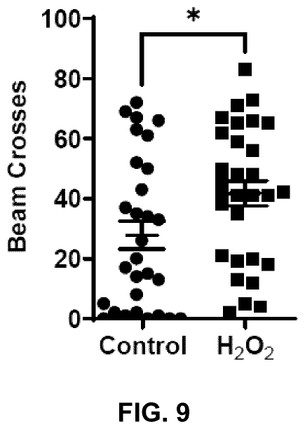

In Drosophila, grooming is a stereotyped, coordinated movement of the forelegs and hindlegs. Grooming is controlled by DA neurotransmission. Increased DA neurotransmission increases grooming. Preliminary data suggest that Xrs significantly promote grooming (FIG. 8A). In a vial containing 11 flies, grooming significantly increases during Xrs stimulation (˜0.2 Gy/sec) with respect to pre Xrs stimulation (FIG. 8A). Locomotion is an elemental behavior regulated by DA neurotransmission across species, including Drosophila. Increased DA neurotransmission increases locomotion2. It is shown that the average velocity enhances during Xrs stimulation (FIGS. 8A-8B, inset). This enhancement translates to a significantly increased distance travelled during Xrs stimulation, as measured by video tracking (FIG. 8B). Placement of the flies in the Xrs chamber without Xrs stimulation did not cause changes in locomotor activity (Student's t-test; p=0.18; n=6; Pre vs. During 30 sec). Finally, considering the hypothesis that Xrs stimulate DA-associated behaviors through H₂O₂ synthesis, it was determined whether H₂O₂, in addition to causing DA release (FIGS. 7A-7B), also regulates locomotion. Flies were fed with food containing either vehicle (control) or H₂O₂ (1%) for 5 hours. Then flies were transferred to locomotion chambers and locomotion was measured by beam crossing detection over a period of 4 hours, after one hour of acclimation. H₂O₂ significantly stimulates locomotion (FIG. 9), consistent with published data.

The most effective dose rates of Xrs stimulation in promoting DA release will be associated with changes in locomotor activity. These changes will be recorded, either by video tracking (FIG. 8A) or by beam crossing (FIG. 9). Next, it will be determined whether the most effective Xrs dose rates also promote grooming. These experiments will be performed as described in FIG. 8A. It will also be determined whether Xrs dose rates that regulate either locomotion or grooming, fail to do so when DA content has been depleted chemically with 3IY (FIGS. 4A-4G) or by RNAi against TH.

The role of dTRPA1 channels responsible for coordinating Xrs stimulation of DA release will be established for Xrs-induced regulation of locomotion and grooming. First, the effective dose rates of Xrs determined to alter locomotion and grooming will be used to irradiate flies fed with dTRPA1 inhibitors. The inhibitors will be dissolved in food or provided to the flies via sucrose feeding (1-5 hr) at effective concentrations, as previously described. Next, these experiments will be performed in flies where the dTRPA1 RNAi(s) target specifically DA neurons, as specified above. As a control, mCherry RNAi targeting DA neurons will be used. It will further be determined whether agonism of dTRPA1 channels with either H₂O₂ (0.1-1%) or (BITC 1-100 μM) dissolved in food (1-5 hr; 1 hr acclimation as in FIG. 9) stimulates locomotion. Once an EC₅₀ of H₂O₂/AITC is established for locomotion, it will be determined whether H₂O₂ also stimulates grooming. These experiments (locomotion and grooming) involving H₂O₂/AITC will be repeated either in flies either fed dTRPA1 inhibitors at concentrations that inhibit Xrs-induced locomotion and grooming, where dTRPA1 RNAi targets DA neurons, or undergoing DA depletion.

It is expected that dTRPA1 RNAi in DA neurons, as well as antagonism of dTRPA1 channels, will inhibit Xrs/H₂O₂ regulation of both locomotion and grooming. It is also expected that DA depletion by 3IY and RNAi targeting TH will impair Xrs/H₂O₂ stimulation of these behaviors.

### Example 3: Experimental Methods

### Lentivirus Design and Stereotaxic Surgery for Viral Expression

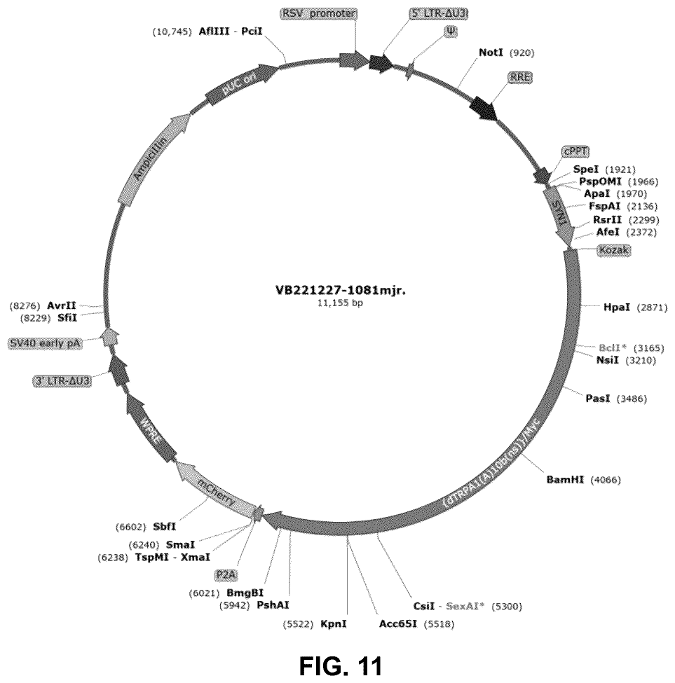

VSV-G pseudotyped third-generation lentivirus carrying dTRPA1(A)10b DNA and associated control were synthesized and packaged by Vectorbuilder. A map of the TRP construct is shown in FIG. 11. An exemplary vector has SEQ ID NO. 1.

All animal procedures and protocols were performed in accordance with regulations of the Institutional Animal Care and Use Committee (IACUC) at the University of Alabama at Birmingham, which operates with accreditation from the Association for Assessment and Accreditation of Laboratory Animal Care (AAALAC). Male C57BL/6J mice at 6-14 weeks of age were group housed on a 12 h light/dark cycle with ad libitum access to food and water.

Stereotaxic surgery was performed with all coordinates taken in reference to bregma. Anesthesia was performed with isoflurane (O₂ flow of 1 L/min, 4% for induction and 2% for maintenance). A thermal heating pad was used to maintain the mouse body temperature for the duration of the surgery. Viruses were injected unilaterally in the substantia nigra (coordinates AP: −3.1, ML: −1.5, DV: −4.5) using a 5 μL Hamilton syringe and a pump. According to company specifications, 4 μL of each virus were injected at a flow rate of 20 nL/min. At the completion of the injection, 5 min were allocated to avoid back flow upon removal of the syringe from the brain. Mice were allowed 3 weeks to express the virus before testing.

### Fast-Scan Cyclic Voltammetry (FSCV) Dopamine Quantification Method

Electrode fabrication: Carbon fiber microelectrodes (CFM) were fabricated by aspirating a carbon fiber (7 μm) into a glass capillary (0.4 mm internal diameter, 0.6 mm outer diameter). Electrodes were pulled to a fine tip using a vertical pipette puller, creating a carbon-glass seal. The exposed carbon fiber was then cut to 50 μm and Nafion (L-Q-1105, Ion Power) was electroplated onto the electrode surface by applying a constant potential (˜1 V) for 30 s.

Stereotaxic surgery for FSCV: Following the administration of urethane anesthesia via i.p. injection, stereotaxic surgery was performed with all coordinates taken in reference to bregma. The CFM was lowered until it was fully immersed in the dorsal striatum (AP: +0.7, ML: −1.5, DV: −2.5 to −3) and compared to a pseudo-Ag/AgCl reference electrode placed in the contralateral hemisphere. A stimulating electrode was also placed in the medial forebrain bundle (AP: −1.58, ML: −1, DV: −4.8). A thermal heating pad was used to maintain mouse body temperature for the duration of the experiment.

All measurements were collected using a Dagan potentiostat, custom built hardware interfaced with PCIe 6431 and PCI 6221 DAC/ADC cards (National Instruments), and a Pine Research headstage. WCCV 3.06 software (Knowmad Technologies) was used to apply the dopamine waveform (−0.4 V to 1.3 V to −0.4 V) at a scan rate of 1000 V s⁻¹. The waveform was cycled at a frequency of 60 Hz for 10 min, then at 10 Hz for 10 min prior to data acquisition.

Dopamine release was induced irradiating the mouse using a Moxtek X-ray source equipped with a custom-built shutter electronically controlled by the acquisition software. Following X-ray induced release experiment, a biphasic electrical stimulation (60 Hz, 360 μA, 2 ms in width) was applied for 2 s through a linear constant current stimulus isolator to electrically evoke dopamine release, to act both as confirmation of striatal localization and to serve as control for quantification purposes.

FSCV processing: FSCV data was filtered and processed using The Analysis Kid and the scikit-learn library. A bandpass, 5^{th} order Butterworth filter was applied with cutoff frequencies of 0.01 Hz and 0.5 Hz to remove baseline drifts and high frequency noise from the signal. Following, a partial least squares regression (PLSR) model (5 components) was trained to predict dopamine concentration from in vitro flow cell FSCV injections (100 nM, 500 nM and 1000 nM, three repetitions per solution) using all the time series of the CV. The PLSR model was then applied to the in vivo FSCV acquisitions to predict the dopamine concentration vs. time traces. The maximum amplitude and area under the curve of the dopamine trace following stimulation were calculated using a local maxima stochastic algorithm and the Simpson rule, implemented in The Analysis Kid.

Statistical analyses: All group-level statistical analyses were performed using the SciPy library (1.10.0) with Python 3.11. The reported p values are two-sided. Univariate statistical methods (independent t-tests) were used to assess the differences maximum amplitude and area under the curve of dopamine concentration traces of mcherry and TRPA1-mcherry mice following stimulation. Statistical significance was defined at a threshold α=0.05.

### ROS Quantification Assays

DCFDA-Cellular ROS assay; The relative levels of basal intracellular ROS were determined using DCFDA-cellular ROS assay. DCFDA (2′,7′-dichlorofluorescin diacetate) is a cell permeable, fluorogenic dye that is used to semi-quantitatively measure hydroxyl, peroxyl and other ROS levels in live cell samples. This assay works based on the diffusion of DCFDA into the cells, which is then deacetylated by cellular esterases to a cell impermeable non-fluorescent compound (2′,7′-dichlorodihydrofluorescein, H2DCF), and then oxidized by ROS into a highly fluorescent compound (2′,7′-dichlorofluorescein, DCF). This signal is detected by microplate reader with excitation at 485 nm and emission at 535 nm. Basal ROS levels are approximated by normalizing the 1 hour signal to the baseline signal.

HyPer-3 biosensor for H₂O₂: HyPer3 probe is a genetically encoded fluorescent indicator for intracellular H₂O₂ since it contains the circularly permuted yellow fluorescent protein (cpYFP) inserted into OxyR. OxyR is a transcription factor from Escherichia coli that senses H₂O₂. In the presence of H₂O₂, a disulfide bond is formed between two cysteine (Cys) residues located in the amino (N) and carboxyl (C) domains of HyPer. This in turn induces a shift in the fluorescence excitation spectrum of cpYFP from 405 nm to 488 nm, corresponding to the protonated and deprotonated form of cpYFP.

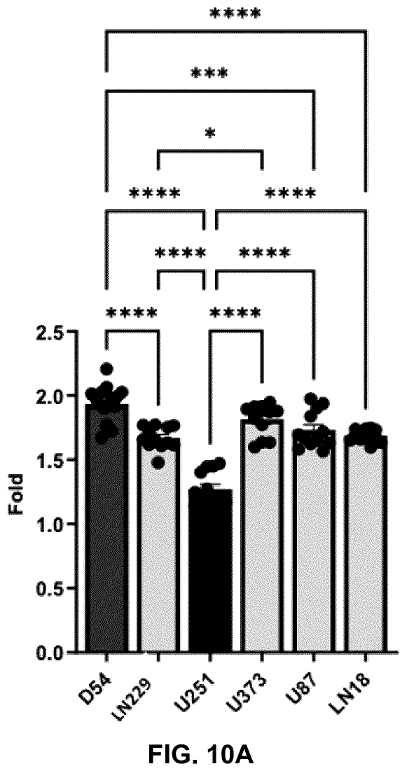

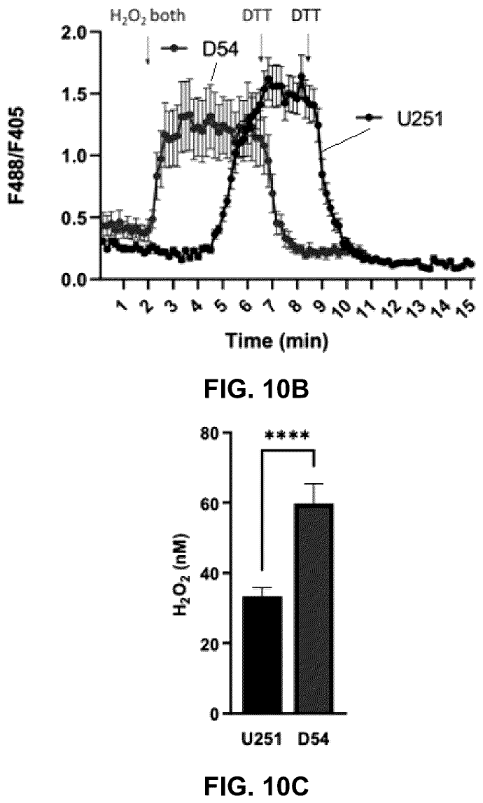

Results indicating differential ROS levels exist between glioblastoma cell lines are shown in FIGS. 10A-10C.

### Immunohistochemistry

After experiments, mice were euthanized and transcardiac perfusion was performed (ice-cold PBS then 4% PFA in PBS). Brain were sliced with a vibratome and the representative free-floating 200 μm-thick coronal sections were permeabilized for 1 h in 0.5% Triton-100/PBS and then blocked in 10% normal goat serum in 0.1% Triton/PBS containing 1% w/v Bovine Serum Albumin (BSA) for 1 h at room temperature. Slices were then incubated in polyclonal rabbit α-mCherry (1:500; Abcam; Cat. #ab167453) overnight at 4° C. and then followed by Alexa Fluor™ 488 Goat anti-Rabbit IgG (1:200; Thermo Fisher Scientific; Cat. #A-11034) for 2 h at room temperature. mCherry immunolabeling was visualized using a Keyence Fluorescence Microscope to confirm expression.

### Example 4: Use of Nanoparticles to Enhance ROS Production

Metal nanoparticles useful herein include gold, platinum, bismuth, tungsten, silver, gadolinium, tantalum, or any combination thereof. Metal oxide nanoparticles useful herein include titanium dioxide (TiO₂), zinc oxide (ZnO), cerium oxide (CeO₂), iron oxides (Fe₃O₄ and/or Fe₂O₃), manganese dioxide (MnO₂), or any combination thereof. Polymeric nanoparticles can encapsulate other materials including, but not limited to, metal salts, photosensitizers, or radiocatalysts, or can serve as functional carriers. Polymeric nanoparticles can be functionalized with specific chemical groups to enhance catalytic surfaces. Examples of suitable polymeric nanoparticles include, but are not limited to poly(lactic-co-glycolic acid) (PLGA), polyethylene glycol (PEG) coated systems for improved circulation, chitosan-based nanoparticles, and conductive polymers including, but not limited to, polypyrrole or polyaniline polymers. Conductive polymers may be useful for charge-transfer purposes in ROS catalysis. Some of these polymers can degrade when exposed to X-rays, causing the release of any payload during irradiation. Doping the loaded nanoparticles can enhance the release of payload. Composite nanoparticles can include core-shell structures (e.g. gold core with TiO₂ shell), hybrid systems (gadolinium-doped ZnO or Fe₃O₄—TiO₂ composites), liposome-based nanocarriers loaded with radiolytic catalysts, or any combination thereof. Nanoparticles from more than one class above can be used together.

The nanoparticles can be administered by intravenous injection for systemic delivery with targeting ligands such as, for example, transferrin, antibodies, or the like. The nanoparticles can be administered intranasally. The nanoparticles can be delivered by localized injection into CNS targets including, but not limited to, the striatum, the ventral tegmental area (VTA), or spinal cord for regional concentration. Convection-enhanced delivery (CED) can be used to deliver the nanoparticles. The nanoparticles can be encapsulated in hydrogel or implantable matrix for sustained release.

Under X-ray irradiation, nanoparticles enhance the local radiation dose due to increased photoelectric and Compton scattering, leading to localized energy deposition. This interaction generates secondary electrons (photoelectrons, Auger electrons) that interact with surrounding water, resulting in hydroxyl radicals, superoxide anions, and hydrogen peroxide. Certain metal oxides including titanium dioxide and zinc oxide can also catalyze the disproportionation or transformation of radicals into more stable ROS like hydrogen peroxide. Nanoparticles can localize ROS production near membrane receptors or ion channels (e.g., dTRPA1) to enhance neuromodulation efficiency.

For embodiments in which nanoparticles are used, the nanoparticles are first administered by a disclosed method. The nanoparticles are targeted via passive (enhanced permeability and retention effect, or EPR) or active (ligand-mediated) targeting to desired neural regions. The nanoparticles are irradiated with focused X-rays as described herein. This activates the nanoparticles and, in some cases, activates secondary electron emission. Water radiolysis occurs and ROS are generated, which in turn activates hydrogen peroxide-sensitive channels and receptors, such as the disclosed virally transfected dTRPA1.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Aguilar JI, et al. Psychomotor impairments and therapeutic implications revealed by a mutation associated with infantile Parkinsonism-Dystonia. E life. 2021; 10.
2. Analysis Kid, The. <analysis-kid.hashemilab.com> Website. Accessed March 28, 2025.
3. Andersson DA, et al. Transient receptor potential A1 is a sensory receptor for multiple products of oxidative stress. J Neurosci. 2008; 28:2485-94.
4. Belovich AN, et al. A network of phosphatidylinositol (4,5)-bisphosphate (PIP2) binding sites on the dopamine transporter regulates amphetamine behavior in Drosophila Melanogaster. Mol Psychiatry. 2019.
5. Boiko N, et al. TrpA1 activation in peripheral sensory neurons underlies the ionic basis of pain hypersensitivity in response to vinca alkaloids. PLOS One. 2017; 12:e0186888.
6. Cabezas-Bratesco D, et al. Sequence and structural conservation reveal fingerprint residues in TRP channels. E life. 2022; 11.
7. Campbell NG, et al. Structural, functional, and behavioral insights of dopamine dysfunction revealed by a deletion in SLC6A3. Proc Natl Acad Sci U S A. 2019; 116:3853-3862.
8. Cartier E, et al. Rare autism-associated variants implicate syntaxin 1 (STX1 R26Q) phosphorylation and the dopamine transporter (hDAT R51W) in dopamine neurotransmission and behaviors. E BioMedicine. 2015; 2:135-146.
9. Chang HY, et al. Overexpression of the Drosophila vesicular monoamine transporter increases motor activity and courtship but decreases the behavioral response to cocaine. Mol Psychiatry. 2006; 11:99-113.
10. Chung KK, et al. Expression and functional properties of TRPM2 channels in dopaminergic neurons of the substantia nigra of the rat. J Neurophysiol. 2011; 106:2865-75.
11. Cichewicz K, et al. A new brain dopamine-deficient Drosophila and its pharmacological and genetic rescue. Genes Brain Behav. 2017; 16:394-403.
12. Connolly K. The social facilitation of preening behaviour in Drosophila melanogaster. Anim Behav. 1968; 16:385-91.
13. Davis RL. Traces of Drosophila memory. Neuron. 2011; 70:8-19.
14. Du EJ, et al. Nucleophile sensitivity of Drosophila TRPA1 underlies light-induced feeding deterrence. E life. 2016; 5.
15. Fernandez RW, et al. Modulation of social space by dopamine in Drosophila melanogaster, but no effect on the avoidance of the Drosophila stress odorant. Biol Lett. 2017; 13.
16. Fricke H. et al. The Reduction of Oxygen to Hydrogen Peroxide by the Irradiation of Its Aqueous Solution with X-Rays. The Journal of Chemical Physics. 1934:556-557.
17. Gibhardt CS, et al. X-ray irradiation activates K+ channels via H₂O2 signaling. Sci Rep. 2015; 5:13861.
18. Grover D, et al. Hydrogen peroxide stimulates activity and alters behavior in Drosophila melanogaster. PLOS One. 2009; 4:e7580.
19. Guntur AR, et al. Drosophila TRPA1 isoforms detect UV light via photochemical production of H2O2. Proc Natl Acad Sci U S A. 2015; 112:E5753-61.
20. Hamilton PJ, et al. De novo mutation in the dopamine transporter gene associates dopamine dysfunction with autism spectrum disorder. Mol Psychiatry. 2013.
21. Hamilton PJ, et al. PIP 2 regulates psychostimulant behaviors through its interaction with a membrane protein. Nat Chem Biol. 2014; 10:582-9.
22. Hockberger PE, et al. Activation of flavin-containing oxidases underlies light-induced production of H2O2 in mammalian cells. Proc Natl Acad Sci U S A. 1999; 96:6255-60.
23. Hong C, et al. TRP Channels as Emerging Therapeutic Targets for Neurodegenerative Diseases. Front Physiol. 2020; 11:238.
24. Klein JS, et al. Radioluminescence in biomedicine: physics, applications, and models. Phys Med Biol. 2019; 64:04TR01.
25. Kobayashi K. Pulse Radiolysis Studies for Mechanism in Biochemical Redox Reactions. Chem Rev. 2019:4413-4462.
26. Landayan D, et al. Satiation state-dependent dopaminergic control of foraging in Drosophila. Sci Rep. 2018; 8:5777.
27. Le Caer S. Water Radiolysis: Influence of Oxide Surfaces on H2 Production under Ionizing Radiation. Water. 2011:235-253.
28. Li F, et al. The connectome of the adult Drosophila mushroom body provides insights into function. E life. 2020; 9.
29. Luo J, et al. TRPA1 mediates sensation of the rate of temperature change in Drosophila larvae. Nat Neurosci. 2017; 20:34-41.
30. Minke B. The history of the Drosophila TRP channel: the birth of a new channel superfamily. J Neurogenet. 2010; 24:216-33.
31. Negishi T, et al. Increased levels of 8-hydroxy-2′-deoxyguanosine in Drosophila larval DNA after irradiation with 364-nm laser light but not with X-rays. Photochem Photobiol. 2007; 83:658-63.
32. Pendleton RG, et al. Effects of tyrosine hydroxylase mutants on locomotor activity in Drosophila: a study in functional genomics. Behav Genet. 2002; 32:89-94.
33. Pitmon E, et al. The D1 family dopamine receptor, DopR, potentiates hind leg grooming behavior in Drosophila. Genes Brain Behav. 2016; 15:327-34.
34. Pizzo AB, et al. The membrane raft protein Flotillin-1 is essential in dopamine neurons for amphetamine-induced behavior in Drosophila. Mol Psychiatry. 2013; 18:824-33.
35. Reddy IA, et al. Bile diversion, a bariatric surgery, and bile acid signaling reduce central cocaine reward. PLOS Biology. 2018; DOI: 10.1371/journal.pbio.2006682.
36. Skikit-learn Library. <scikit-learn.org/stable/> Website. Accessed March 28, 2025.
37. SciPy Library. <scipy.org> Website. Accessed March 28, 2025.
38. Shekar A, et al. Syntaxin 1 Ser(14) phosphorylation is required for nonvesicular dopamine release. Sci Adv. 2023; 9:eadd8417.
39. Terada Y, et al. Structure-Activity Relationship Study on Isothiocyanates: Comparison of TRPA1-Activating Ability between Allyl Isothiocyanate and Specific Flavor Components of Wasabi, Horseradish, and White Mustard. J Nat Prod. 2015; 78:1937-41.
40. Ton HT, et al. Identification of a putative binding site critical for general anesthetic activation of TRPA1. Proc Natl Acad Sci U S A. 2017; 114:3762-3767.
41. Veal EA, et al. Hydrogen peroxide sensing and signaling. Mol Cell. 2007; 26:1-14.
42. Wada M, et al. Responses to transient receptor potential (TRP) channel agonists in Chlamydomonas reinhardtii. Biol Open. 2020; 9.
43. Wicker-Thomas C et al. Interaction of dopamine, female pheromones, locomotion and sex behavior in Drosophila melanogaster. J Insect Physiol. 2008; 54:1423-31.
44. Yellman C, et al. Conserved and sexually dimorphic behavioral responses to biogenic amines in decapitated Drosophila. Proc Natl Acad Sci U S A. 1997; 94:4131-6.
45. Zhang J, et al. Endogenous ion channels expressed in human embryonic kidney (HEK-293) cells. Pflugers Arch. 2022; 474:665-680.
46. Zhong L, et al. Thermosensory and nonthermosensory isoforms of Drosophila melanogaster TRPA1 reveal heat-sensor domains of a thermoTRP Channel. Cell Rep. 2012; 1:43-55.

What is claimed is:

1. A method for neuromodulation in a subject, the method comprising:
virally transfecting one or more neurons in the subject with a gene encoding dTRPA1(A)10b, thereby causing dTRPA1(A)10b channels to be expressed in the one or more neurons;
delivering X-ray radiation to the one or more neurons;
wherein the X-ray radiation generates H₂O₂ via radiolysis of water and wherein the H₂O₂ activates the dTRPA1(A)10b channels; thereby increasing intercellular calcium and releasing one or more neurotransmitters.

2. The method of claim 1, wherein from about 1 mGy to about 500 mGy of X-ray radiation are delivered to the subject.

3. The method of claim 1, wherein the X-ray radiation is delivered with an exposure time of from about 1 ms to about 25 s.

4. The method of claim 1, wherein the X-ray radiation has a frequency of from about 0.1 Hz to about 100 Hz.

5. The method of claim 1, wherein the subject comprises an isolated plurality of cells, an isolated organ, or an animal subject.

6. The method of claim 5, wherein the isolated plurality of cells comprise one or more neurons, wherein the one or more neurons are mammalian neurons.

7. The method of claim 5, wherein the isolated organ comprises a brain.

8. The method of claim 5, wherein the animal subject comprises Drosophila, a human, mouse, rat, guinea pig, hamster, rabbit, cat, dog, cattle, horse, swine, goat, sheep, or non-human primate.

9. The method of claim 6, wherein the one or more neurons comprise neurons in the central nervous system, neurons in the peripheral nervous system, or any combination thereof.

10. The method of claim 1, wherein the one or more neurotransmitters comprise dopamine, serotonin, norepinephrine, or any combination thereof.

11. The method of claim 1, wherein the method is used to treat a neurological disease or disorder in a subject.

12. The method of claim 11, wherein the neurological disease or disorder comprises spinal cord injury, amyotrophic lateral sclerosis (ALS), or Parkinson's disease.

13. The method of claim 1, wherein the method is used to modulate a symptom of a disease or disorder in a subject.

14. The method of claim 13, wherein the symptom is micturition dysfunction resulting from spinal cord injury, neurogenic bladder, or another cause of lower urinary tract dysfunction.

15. The method of claim 1, further comprising administering nanoparticles to the one or more neurons, wherein the nanoparticles enhance generation of one or more reactive oxygen species, and wherein the nanoparticles comprise metal nanoparticles, metal oxide nanoparticles, polymeric nanoparticles, composite nanoparticles, or any combination thereof.

16. A method for treating brain cancer in a subject, the method comprising:
virally transfecting one or more brain cancer cells in the subject with a gene encoding dTRPA1(A)10b;
delivering X-ray radiation to the one or more brain cancer cells;
wherein the X-ray radiation generates H₂O₂ via radiolysis of water and wherein the H₂O₂ activates the dTRPA1(A)10b channels; thereby increasing intercellular calcium and selectively inducing apoptosis.

17. The method of claim 16, wherein from about 1 mGy to about 500 mGy of X-ray radiation are delivered to the subject.

18. The method of claim 16, wherein the X-ray radiation is delivered with an exposure time of from about 1 ms to about 25 s.

19. The method of claim 16, wherein the X-ray radiation has a frequency of from about 0.1 Hz to about 100 Hz.

20. The method of claim 16, further comprising administering nanoparticles to the one or more neurons, wherein the nanoparticles enhance generation of one or more reactive oxygen species, and wherein the nanoparticles comprise metal nanoparticles, metal oxide nanoparticles, polymeric nanoparticles, composite nanoparticles, or any combination thereof.
